(19) United States
(12) Patent Application Publication
(10) Pub. No.: US 2026/0007562 A1
(43) Pub. Date: Jan. 8, 2026

(54) Flexible Robotic Actuator, Apparatus, System and Method Thereof

(71) Applicant: THE HONG KONG POLYTECHNIC UNIVERSITY, Hong Kong (CN)

(72) Inventors: Ho Lam HEUNG, Hong Kong (CN); Sheung Mei Shamay NG, Hong Kong (CN); Wai Lung Thomson WONG, Hong Kong (CN)

(21) Appl. No.: 18/997,206

(22) PCT Filed: Jul. 28, 2023
(86) PCT No.: PCT/CN2023/109850

§ 371 Date: Jan. 21, 2025

Related U.S. Application Data
(60) Provisional application No. 63/370,402, filed on Aug. 4, 2022.

(51) Int. Cl. A61H 1/02; A61N 1/36; B25J 9/00; B25J 9/14; F15B 15/10

(52) U.S. Cl. CPC A61H 1/0288; A61N 1/36003; B25J 9/0006; B25J 9/142; F15B 15/103; A61H 2201/10; A61H 2201/1246; A61H 2201/1635; A61H 2201/165; A61H 2201/1659; A61H 2201/5069

(57) ABSTRACT

One embodiment provides a flexible robotic actuator for assisting a body part of a subject. The flexible robotic actuator comprises a soft body and at least one chamber. The soft body has a first side and a second side opposite the first side, and includes a patterned section on the first side. The at least one chamber is defined by the soft body and operatively driven by a pressurized fluid such that the soft body bends towards the patterned section and the bending angle of the soft body is limited by the patterned section.

### CROSS-REFERENCE TO RELATED APPLICATIONS

This application claims priority to the U.S. provisional patent application Ser. No. 63/370,402, filed Aug. 4, 2022, entitled “Flexible Robotic Actuator for Upper Limb Rehabilitation”, hereby incorporated herein by reference as to its entirety.

### FIELD OF THE INVENTION

The invention relates generally to flexible robotic actuator, apparatus, system, and method thereof for assisting a body part of a subject, such as a human.

### BACKGROUND

The following discussion of the prior art is intended to present the invention in an appropriate technical context and allow its advantages to be properly appreciated. Unless clearly indicated to the contrary, however, reference to any prior art in this specification should not be construed as an express or implied admission that such art is widely known or forms part of common general knowledge in the field.

Robotics devices have been widely adopted to assist a subject, such as a human. For example, robotic devices can assist in the rehabilitation of upper limb function, specifically for hands and wrists. As an example, spasticity, a medical condition that is characterized by an excessive tone in muscles, affects natural movement of body joints after a stroke. Potential solutions for assisting in the movement of spastic joints, e.g., fingers or wrists, after stroke have been seen in the field of wearable robotics. These robotics devices or robots are designed to overcome joint spasticity and provide direct assistance with finger and wrist movement. These hand and wrist robots are mostly constructed with powerful electric motors and rigid metal frameworks, which are bulky, hard to further reduce the weight, and may pose dangers to patients due to over-powered. These devices are also mostly developed for laboratory settings. They are tethered to giant power sources, and therefore become cumbersome and unattractive to be used outside the clinic or laboratory. Therefore, there is a need in the industry of robotic technologies that offer advantages in interacting with and assisting in a subject in the functionalities in a certain body part.

It is an object of the present invention to overcome or substantially ameliorate one or more of the disadvantages of prior art, or at least to provide a useful alternative.

### SUMMARY

In one aspect of the invention there is provided a flexible robotic actuator for assisting a body part of a subject. The flexible robotic actuator comprises a soft body and at least one chamber. The soft body has a first side and a second side opposite the first side, and includes a patterned section on the first side. The at least one chamber is defined by the soft body and operatively driven by a pressurized fluid such that the soft body bends towards the patterned section in a first direction and the bending angle of the soft body is limited by the patterned section.

In another aspect of the invention there is provided a flexible robotic apparatus for assisting a body part of a subject. The flexible robotic apparatus comprises a soft base wearable onto the body part of the subject and at least one flexible robotic actuator according to one or more aspects of the invention. The at least one flexible robotic actuator is configured to be secured to the soft base. The soft base includes at least one fluid inlet in fluid communication with the at least one chamber for receiving the pressurized fluid received from an external fluid source, and at least one data port electrically communicating with an external electrical system such that at least one parameter associated with the at least one flexible robotic actuator is monitored.

In a further aspect of the invention there is provided a flexible robotic system for assisting a body part of a subject. The flexible robotic system comprises a flexible robotic apparatus and a control system. The flexible robotic apparatus includes a soft base wearable onto the body part of the subject and at least one flexible robotic actuator configured to be secured to the soft base. Each of the at least one flexible robotic actuator includes a soft body and at least one chamber defined by the soft body. The soft body includes a patterned section on one side and is configured to operatively bend towards the patterned section when the at least one chamber is driven by a pressurized fluid. The control system is in fluid communication with the at least one chamber of each of the at least one flexible robotic actuator for controlling injection of the pressurized fluid into the at least one chamber, and in electrical communication with the flexible robotic apparatus such that operation of the at least flexible robotic actuator is electrically controlled.

In yet a further aspect of the invention there is provided a method for assisting a body part of a subject. The method comprises: providing a flexible robotic apparatus, the flexible robotic apparatus including a soft base wearable onto the body part of the subject and a flexible robotic actuator, the flexible robotic actuator being secured to the soft base, the flexible robotic actuator including a soft body and at least one chamber defined by the soft body, the soft body including a patterned section on one side and being provided with a plurality of ring constraints surrounding the at least one chamber, the plurality of ring constraints being provided with a plurality of anchor structures on the other side of the soft body opposite to the one side; and injecting a pressurized fluid into the at least one chamber such that the soft body bends towards the patterned section and the bending angle of the soft body is limited by the patterned section.

Other example embodiments are discussed herein.

### BRIEF DESCRIPTION OF THE DRAWINGS

Embodiments of the invention will now be described, by way of example only, with reference to the accompanying drawings in which:

FIG. 1 illustrates a flexible robotic apparatus including a flexible robotic actuator according to certain embodiments of the present invention, where the flexible robotic apparatus is exemplified as a robotic wrist.

FIG. 2A illustrates the flexible robotic actuator of FIG. 1, where the flexible robotic actuator is in a non-bending state.

FIG. 2B illustrates the structure of the flexible robotic actuator of FIG. 2A from a cross-sectional perspective.

FIG. 2C illustrates the flexible robotic actuator of FIG. 2A, where the flexible robotic actuator is in a bending state.

FIG. 3 illustrates the Range-of-Motion (ROM) characteristic of a flexible robotic actuator with respect to the pressure-angle relationship according to certain embodiments of the present invention.

FIG. 4A illustrates a flexed wrist of a patient suffering from spasticity according to certain embodiments of the present invention.

FIG. 4B illustrates a flexible robotic actuator extends the wrist of FIG. 4A to a neutral position.

FIG. 4C illustrates a full extension of the wrist of FIG. 4A upon pressurization of the flexible robotic actuator.

FIG. 5 illustrates a flexible robotic apparatus including flexible robotic actuators according to certain embodiments of the present invention, where the flexible robotic apparatus is exemplified as a robotic hand.

FIG. 6A illustrates a finger actuator according to certain embodiments of the present invention.

FIG. 6B illustrates the internal structure of the finger actuator of FIG. 6A in a bending state upon pressurization.

FIG. 6C illustrates the internal structure of the finger actuator of FIG. 6A in an extending state upon pressurization.

FIG. 7 illustrates the ROM characteristic of the finger actuator of FIG. 6A with respect to the pressure-angle relationship in the bending state.

FIG. 8 illustrates the ROM characteristic of the finger actuator of FIG. 6A with respect to the pressure-angle relationship in the extending state.

FIG. 9 illustrates the internal structure of a hand base of a robotic hand having a clamping structure to secure finger actuators into position according to certain embodiments of the present invention.

FIG. 10A illustrates a control system of a flexible robotic system according to certain embodiments of the present invention.

FIG. 10B illustrates an internal configuration of the control system of FIG. 10A.

FIG. 11 illustrates a flexed finger of a patient suffering from spasticity.

FIG. 12 illustrates a control scheme for a finger actuator depending on the severity of muscle spasticity measured by Modified Ashworth Scale (MAS) according to certain embodiments of the present invention.

FIG. 13 illustrates the calibration of joint movement intention from a subject according to certain embodiments of the present invention.

FIG. 14 illustrates measuring the joint angle using a thin-film flex sensor according to certain embodiments of the present invention.

FIG. 15 illustrates a method of assisting a subject in wrist or finger extension with the usage of electric current.

FIG. 16A illustrates a soft actuator according to certain embodiments of the present invention.

FIG. 16B illustrates separated actuation chambers of the soft actuator of FIG. 16A.

FIG. 16C illustrates an exploded view of the soft actuator of FIG. 16A.

FIG. 17A illustrates a flexible robotic system according to certain embodiments of the present invention, where the flexible robotic system includes a flexible robotic apparatus and a control box, and the flexible robotic apparatus is exemplified as a robotic hand.

FIG. 17B illustrates an exploded view of the robotic hand of FIG. 17A.

FIG. 17C illustrates the configuration of the control box of FIG. 17A.

FIG. 17D illustrates the control logic for the robotic hand of FIG. 17A for facilitating hand closing and opening movement.

FIG. 18A shows a finite element method (FEM)-simulated free space bending of a soft actuator at a pressure input of 300 kPa according to certain embodiments of the present invention.

FIG. 18B shows a FEM-simulated contact force bending of a soft actuator at a pressure input of 300 kPa when the soft actuator contacts an object according to certain embodiments of the present invention.

FIG. 19A illustrates an example setup for actuator characterization regarding free space bending for a soft actuator according to certain embodiments of the present invention.

FIG. 19B illustrates an example setup for actuator characterization regarding contact force bending for a soft actuator according to certain embodiments of the present invention.

FIG. 20 illustrates a soft actuator's dimensions and torque generated around the fulcrum O in a bending state at the tip of the soft actuator during free space bending and the contact with objects for purpose of actuator modelling according to certain embodiments of the present invention.

FIG. 21A shows a pressure-angle relationship of the soft actuator for index, middle, and ring fingers according to certain embodiments of the present invention.

FIG. 21B shows a pressure-angle relationship of the soft actuator for a small finger according to certain embodiments of the present invention.

FIG. 21C shows a pressure-angle relationship of the soft actuator for a thumb according to certain embodiments of the present invention.

FIG. 22A shows three soft actuators with a length of chamber of 80 mm, 60 mm, and 40 mm respectively.

FIG. 22B illustrate a 10-degree angle position with respect to the proximal end of the soft actuator.

FIG. 22C illustrate a 40-degree angle position with respect to the proximal end of the soft actuator.

FIG. 22D shows a pressure-force relationship for the three soft actuators of FIG. 21A at a 10-degree angular position.

FIG. 22E shows a pressure-force relationship for the three soft actuators of FIG. 21A at a 40-degree angular position.

FIG. 23A illustrates actuator tip force during the pinch of an index finger for a card according to certain embodiments of the present invention.

FIG. 23B illustrates actuator tip force during the pinch of an index finger for a wooden box according to certain embodiments of the present invention.

FIG. 24A illustrates an equivalent model of a soft actuator for analyzing the grip force at Metacarpophalangeal (MCP) and Proximal Interphalangeal (PIP) joints respectively according to certain embodiments of the present invention.

FIG. 24B illustrates the estimated grip force and measured joint angle during palm grasp of a bottle.

FIG. 24C illustrates the estimated grip force and measured joint angle during end grasp a pen.

### DETAILED DESCRIPTION

The present invention will now be described with reference to the following examples which should be considered in all respects as illustrative and non-restrictive. In the Figures, corresponding features within the same embodiment or common to different embodiments have been given the same or similar reference numerals.

Throughout the description and the claims, the words “comprise”, “comprising”, and the like are to be construed in an inclusive sense as opposed to an exclusive or exhaustive sense; that is to say, in the sense of “including, but not limited to”.

Furthermore, as used herein and unless otherwise specified, the use of the ordinal adjectives “first”, “second”, etc., to describe a common object, merely indicate that different instances of like objects are being referred to, and are not intended to imply that the objects so described must be in a given sequence, either temporally, spatially, in ranking, or in any other manner.

Example embodiments relate to flexible robotic actuator, apparatus, system, and method thereof for assisting a body part of a subject. The subject may be a human, such as a patient, or an animal, such as a pet animal, e.g., a dog or a cat. The body part of the subject may be an upper limb, such as a wrist or a hand, or other parts, such as a leg, a foot. The body part may be compromised due to various reasons, such as trauma, incomplete spinal cord injuries, stroke, multiple sclerosis, muscular dystrophies, or cerebral palsy. Therefore, the body part may need medical rehabilitation or assistant to restore part or the whole of its function. The body part may be not compromised, but still it is preferable to be provided with certain assistance so that it can function desirably.

Many existing systems have various disadvantages, such as bulky, inconvenient to carry, not compliant for the subject, unsatisfactory lifespan, uncontrollable output control to the joints of the subject, etc. For example, many existing systems for medical rehabilitation include heavy and bulky electric motors. Further, for example, existing robotic hands lack a mathematical way to quantify the grip force generated for better controlling the grasp of objects during the performance of activities of daily living (ADL).

Example embodiments solve one or more of these problems associated with the existing systems and provide technical solutions with new designs. According to one or more embodiments, movement of the subject's body part, such as fingers and wrist, is controlled by one or more flexible robotic actuators driven by a pressurized fluid rather than an electric motor, which achieves a much more lightweight and compact design. The pressurized fluid may be air, gas, or liquid, such as water or heavy oil based hydraulic fluid (glycol ethers, organophosphate ester, polyalphaolefin, propylene glycol, or silicone oils, or the like).

One or more embodiments provide a light weight, portable flexible robotic actuator that is compliant with human tissues. The compliance between the actuator and human tissues brings to a minimal harm to the human, such as patients. For example, a portable apparatus incorporating the flexible robotic actuator may allow a patient to wear it like a normal brace around the wrist, which the existing bulky lab-based device cannot offer.

One or more embodiments provide a flexible robotic actuator, apparatus, or system that reduces the size of traditional exoskeletons from huge size to the size comparable with a human, and can satisfactorily restore functions (such as flexion, extension, or the like) of a certain body part (such as fingers, wrist, legs, feet, etc.) of a human during rehabilitation training.

One or more embodiments provide a muscle stimulator appliable to muscles, thereby strengthening the ability of muscles, which is absent in the existing rehabilitation exoskeleton. For example, the muscle stimulator may stimulate the upper limb to facilitate a better movement. In some embodiments, electric current is generated as a muscle stimulator.

One or more embodiments provide a soft wearable robotic hand with active control of finger flexion and extension through an elastomeric-based bi-directional soft actuator. This actuator bends and extends by pneumatic actuation at lower air pressure, and a flex sensor embedded inside the actuator measures the angles of the fingers in real-time. The robotic hand facilitates hand opening and closing by the wearer and successfully assists with grasping objects with sufficient force for ADL-related tasks.

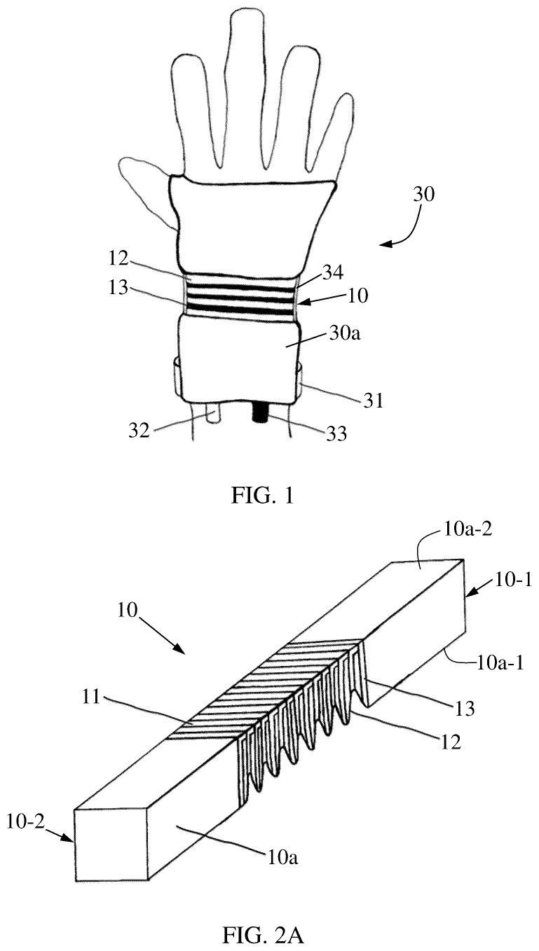

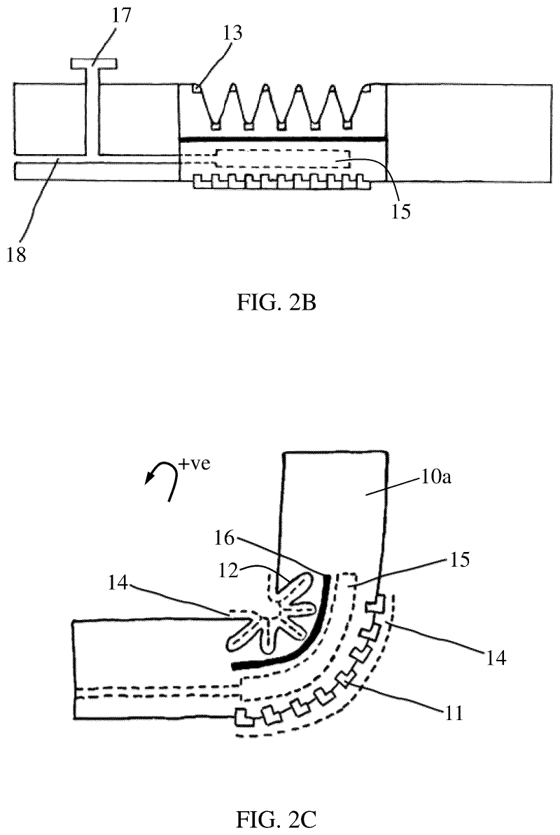

With reference to FIGS. 1, 2A, 2B, and 2C, a flexible robotic apparatus is exemplified as a robotic wrist 30 that comprises a flexible robotic actuator 10. The robotic wrist 30 is wearable on a subject's wrist to aid in movement of the wrist. The subject may be a human, such as a patient whose wrist is compromised and needs assistance.

The robotic wrist 30 may be worn on a human wrist 34 via a wrist band 31. The wrist band 31 forms part of a soft base 30a that may have various mechanical and/or electrical features. The wrist band 31 may be made of one or more materials, such as titanium alloy, nylon, plastics, or carbon fibre composites, such that the wrist band 31 can be properly worn on the human wrist 34. The soft base 30a may be provided with at least one fluid inlet 33 for receiving fluid from an external fluid source and at least one data port 32 that electrically communicates with an external electrical system such that the status of the robotic wrist 30 can be monitored, and/or the operation of the robotic wrist 30 can be controlled.

The flexible robotic actuator 10 comprises a soft body 10a and at least one chamber 15 defined by the soft body 10a. The soft body 10a has a first side or face 10a-1 and a second side or face 10a-2 opposite the first side 10a-1. The soft body 10a includes a patterned section 12 disposed on the first side 10a-1. The patterned section 12 may have a zigzag or waved or toothed pattern.

The soft body 10a has a first end 10-1 and a second end 10-2. The soft body 10a may be formed as an elongated body with a longitudinal axis in parallel to a direction extending from the first end 10-1 towards the second end 10-2. The soft body 10a can be deformed. For example, the soft body 10a may be bent when it is actuated or driven.

The chamber 15 is configured to be operatively driven by a pressurized fluid such that the soft body 10a bends towards the patterned section 12 (the direction of bending may be termed as a first direction) as shown in FIG. 2C and the bending angle of the soft body 10a is limited by the patterned section 12. For example, the pressurized fluid, such as air, can be injected into the chamber 15 via a channel 18 (FIG. 2B). A regulator 17 may be provided to adjust the channel 18. The regulator 17 may control the opening or closing of the channel 18, thereby enabling or disabling the flowing of the fluid therein. The regulator 17 may adjust the cross-sectional area of the channel 18, thereby adjusting the flow rate within the channel 18. The regulator 17 may be designed properly, such as including a valve and a knob that operates the valve. As a result, the pressure in the chamber 15 can be adjusted via the regulator 17, thereby adjusting the bending torque exerted on the soft body 10a.

A plurality of ring constraints 13 may be provided for (such as disposed onto or embedded within) the soft body 10a and surrounding the chamber 15 for restricting axial expansion of the chamber 15 when the chamber 15 is driven by the pressurized fluid. For example, the stiffness of the ring constraints 13 can be higher than the stiffness of the soft body 10a such that the restricting effect can be enhanced. For example, the soft body 10a may be made of one or more materials, such as rubber, silicone, plastics, paper, or the like, that are pliant and can undergo desirable deformation. The ring constraints 13 may be made of one or more materials, such as titanium alloy, nylon, plastic, or carbon fibre composites, or the like, that are lightweight and hard to be broken by the fluid pressure inside the chamber 15 upon pressurization.

Stiffness may be measured in terms of elastic modulus and durometer. The elastic modulus and durometer of the ring constraints 13 may be much larger than that of the soft body 10a. For example, when the ring constraints 13 is made of metal, the elastic modulus is generally larger than 100 Gpa (e.g., Steel: around 200 GPa; Titanium: around 110 GPa; Aluminium: around 69 GPa; Nickel: around 210 GPa: Iron: around 170 GPa; Molybdenum: around 330 GPa). When the ring constraints 13 is made of plastics, the elastic modulus is generally around or less than 10 GPa (e.g., Epoxy Resin: around 5 GPa; Polyester Resin: around 3.3 GPa; Phenolic Resin: around 9 GPa). Material making the soft body 10a may be an elastomer, for which the elastic modulus is generally non-linear or less than 10 MPa (i.e., less than 0.01 GPa), or even lower than 100 kPa (i.e., less than 0.1 MPa).

Further, the plurality of ring constraints 13 may be provided with a plurality of anchor structures 11 on the second side 10a-2 for limiting bending of the soft body 10a towards a second direction opposite the first direction. The anchor structures 11 may be made of rigid material or materials. In some embodiments, because of the patterned section 12 and the anchor structures 11, the bending angle of the soft body 10a can be limited substantially in a range from 0 degree to 90 degree. For example, when the flexible robotic actuator 10 is brought to zero degree (that is, the soft body 10a is straight and not bent), the anchor structures 11 are tightly closed and packed with each other. The legs of the anchor structures 11 collide with each other, which restricts the axial deformation of the chamber 15 when the flexible robotic actuator 10 tries to bend to less than zero degree, thereby preventing the bending angle of being less than zero degree. The patterned section 12 is designed to be fully compressed when the bending angle of the soft body 10a is 90 degree such that any further bending will be prevented. 90 degree is an example bending angle that is specifically set for certain applications, such as in the context of wrist and hand, as the range of motion of wrist and individual finger joints generally typically does not exceed 90 degrees. It will be appreciated that the patterned section 12 may be designed to allow a bending angle larger than 90 degree for the soft body 10a.

A plate member 16 may be embedded within the soft body 10a and disposed between the patterned section 12 and the chamber 15 for facilitating the bending of the soft body 10a. The plate member 16 may have a thickness of less than 1 mm. The plate member 16 may be made of one or more materials, such as plastics, metal, or paper. For example, when the chamber 15 is inflated, the ring constrains 13 will restrict the radial expansion of the chamber 15 and allow only axial elongation to occur. The plate member 16 will put further limitations on the axial elongation at the region around the plate member 16. Eventually, upon inflating the chamber 15, an enhanced bending motion is created for the flexible robotic actuator 10 towards the patterned section 12.

As illustrated in FIG. 2C, an elastic sleeve 14 may be provided for enclosing at least a part of the soft body 10a for improving securing of the ring constraints 13 onto the soft body 10a. For example, the elastic sleeve 14 may cover the peripheral surface of the soft body 10a to secure the ring constraints 13 in position. This can avoid or mitigate unwanted displacement of the ring constraints 13 over time.

Preferably, the durometer of the elastic sleeve 14 is much smaller than the durometer of the soft body 10a such that the elastic sleeve 14 accommodates deformation of the soft body 10a. In this way, coverage of the elastic sleeve 14 does not add significant stiffness to the soft body 10a, thereby avoiding impeding the deformation (such as bending) of the soft body 10a upon fluid injection into the chamber 15. The elastic sleeve 14 may be made of one or more materials, such as rubber, silicone, plastics, or paper, that are pliant and can undergo large deformation. When the elastic sleeve 14 is an elastomer, value of elongation at break can be greater than 300%. The durometer for the soft body 10a and the elastic sleeve 14 may be, but not limited to, Shore 00-10, 20, 30, 40, and 50 or Shore A-10, 20, 30, 40, 50, 60, 70, 80, and 90.

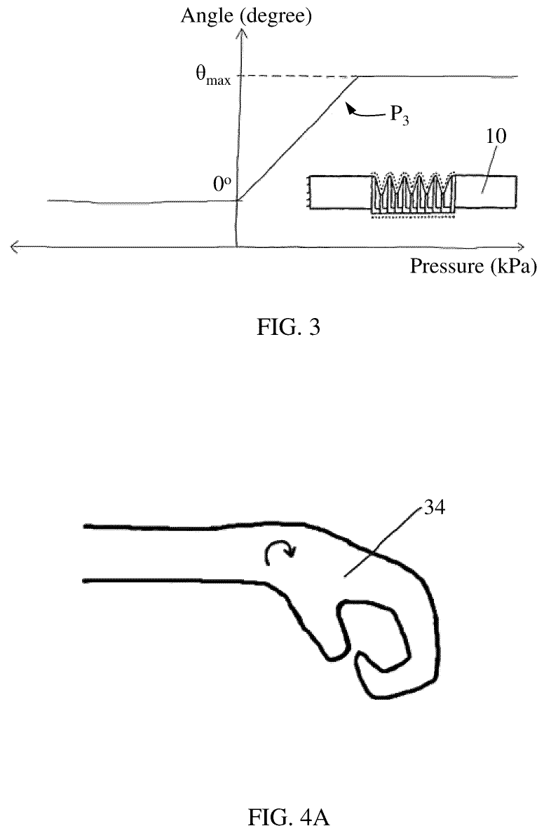

Referring to FIG. 3, the Range-of-Motion (ROM) characteristic of the flexible robotic actuator 10 with respect to the pressure-angle relationship is illustrated. ROM refers to the extent or limit to which a part of the body can be moved around a joint or a fixed point. In this embodiment, when the chamber 15 is actuated or driven under fluid pressure P₃, ROM of the flexible robotic actuator 10 is limited within 0 degree) (0° to θₘₐₓ (which is 90 degree in this example). The anchor structures 11 prevents the bending of the flexible robotic actuator 10 from being less than 0 degree. In the meantime, the patterned section 12 is specifically shaped and sized to only allow a maximum bending of 90 degree for the flexible robotic actuator 10 when the patterned section 12 is fully folded. Regardless of the pressure and/or externally flexing of the flexible robotic actuator 10 by an external force, its ROM always stays within the specified range. It will be appreciated that, however, this is for illustrative purpose only. In some embodiments, the flexible robotic actuator may be designed such that the bending angle can be lower than 0 degree or larger than 90 degree.

Stroke patients typically suffer from wrist contracture mainly due to weakened power in the Extensor Digitorium (ED) muscles and spasticity in the Flexor Digitorium (FD) muscles. They have difficulty in actively and voluntarily control their wrist extension movement. In stroke rehabilitation, restoring the ability of wrist extension is the prerequisite of regaining normal hand function as the ED muscles are also essential for finger extension movement. According to some embodiments, the robotic wrist 30 provides assistance to stroke patients for assisting in their performance of wrist extension.

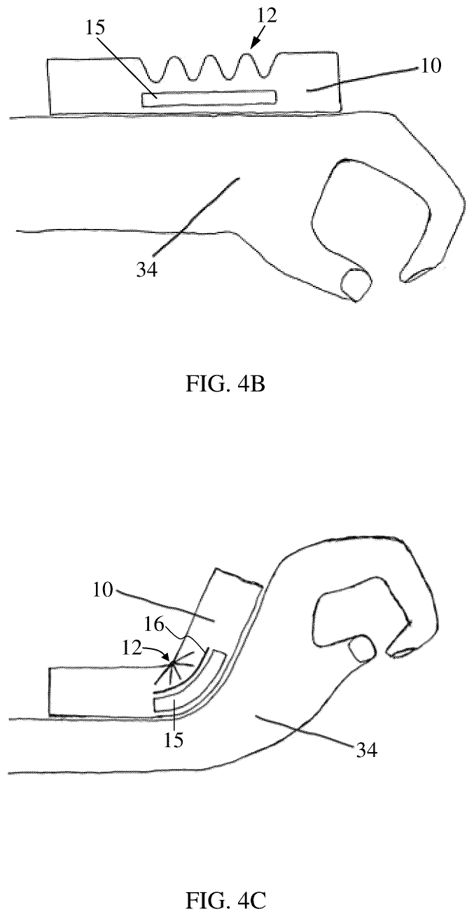

FIGS. 4A, 4B, and 4C show examples of the wearable robotic wrist 30 utilizing an example flexible robotic actuator 10 to extend a flexed human wrist 34. When the chamber 15 is filled with the pressurized fluid, it will drive the soft body to bend, where the bending angle depends largely on the pressure within the chamber 15. The bending of the flexible robotic actuator 10 will bring the human wrist 34 to move together, thereby achieving the natural function of the human wrist 34.

Specifically, as illustrated in FIGS. 4B and 4C, orientation of the flexible robotic actuator 10 is configured such that the anchor structures 11 (not shown, on the face opposite to the patterned section 12) is proximally facing the back side of the human wrist 34, such that the patterned section 12 is away from the human wrist 34. When the robotic wrist 30 is worn on the human wrist 34, the anchor structures 11 can directly straighten the flexed human wrist 34 to maintain it in a neutral position without relying on pressurization of the flexible robotic actuator 10. Once applying a pressurized fluid to the chamber 15, a bending force is generated by the fluid pressure P₃ (FIG. 3) within the chamber 15 to directly extend the human wrist 34. The fluid pressure P₃ may be adjusted by the regulator 17 to control the bending torque once the ROM is within the range from 0 degree to 90 degree. Upon full bending of the soft body 10a, i.e., the bending angle being 90 degree, a maximum bending moment, such as 5 N-m, may be thereby generated by the flexible robotic actuator 10 as an average of wrist extension torque in human. Further increase of fluid pressure P₃ in the chamber 15 can no longer increase the output torque after the bending angle reaches 90 degree.

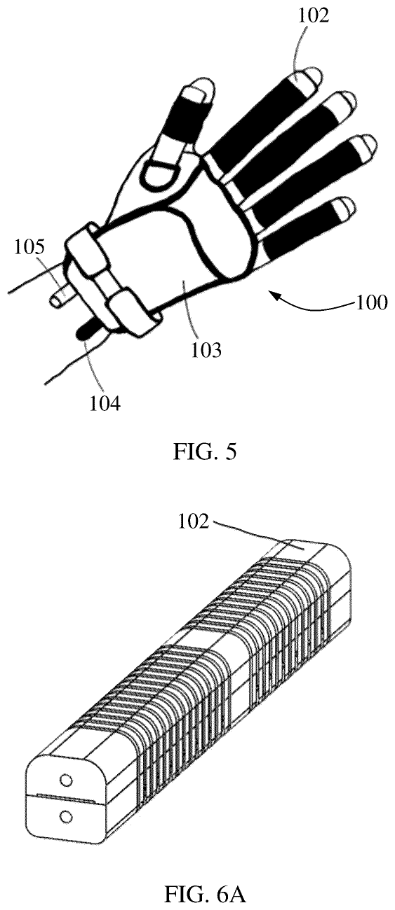

FIG. 5 illustrates a flexible robotic apparatus according to certain embodiments of the present invention, where the flexible robotic apparatus is exemplified as a robotic hand 100. The robotic hand 100 is wearable on a subject's hand to aid in movement of the hand. The subject may be a human, such as a patient whose hand is compromised that needs assistance.

In the present embodiments, the robotic hand 100 includes a soft base or hand base 103 that is wearable onto the subject's hand, such as a human hand. Two flexible robotic actuators 10 are connected in series to form a finger actuator 102 that is secured to the hand base 103. Five finger actuators 102 are incorporated (such as mounted or installed) into the robotic hand 100 for controlling the thumb, index finger, middle finger, ring finger and small finger respectively. Similar to the robotic wrist 30, a fluid inlet 104 and a data port 105 are provided in the hand base 103 for delivering fluid to the finger actuators 102 through a channel 207 and communication with an external electrical system, such as transmitting signal of bending angle measured by a flexible thin-film angle sensor 205, respectively.

Each finger actuator 102 in the present embodiments is illustrated to include two flexible robotic actuators 10 connected in series. This is for illustrative purpose only. It will be appreciated that in some embodiments, each finger actuator may consist of a single flexible robotic actuator, or may include three or more flexible robotic actuators, where these flexible robotic actuators may be connected in series, or in parallel, or combination thereof. The finger actuator is a collection of one or more flexible robotic actuators connected in a specified manner. In this sense, the finger actuator itself is a flexible robotic actuator or a collection of flexible robotic actuators.

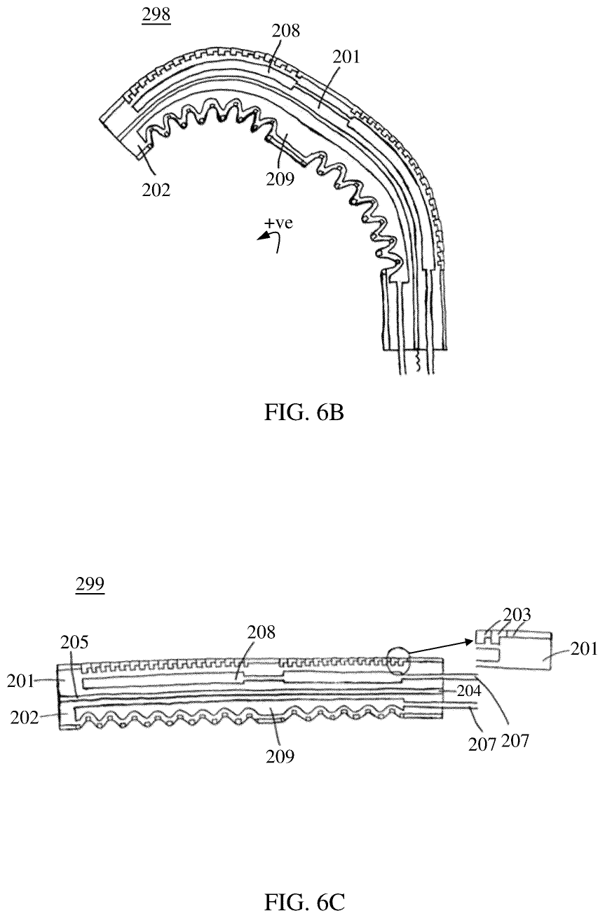

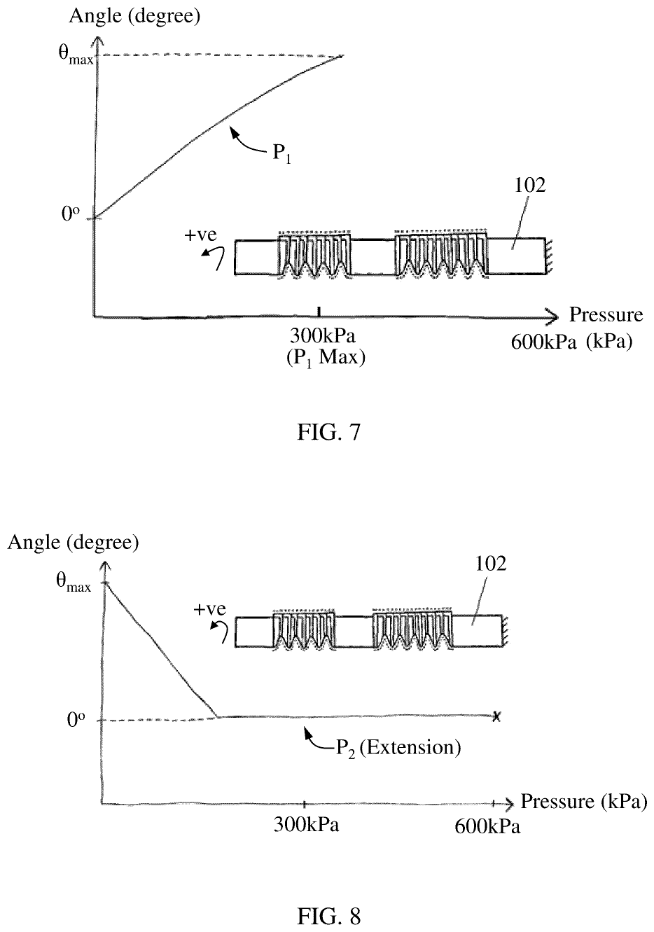

Referring to FIGS. 6A, 6B, 6C, 7, and 8, detailed structure of the flexible robotic actuator 10 included in the finger actuator 102 of FIG. 5 is illustrated. The flexible robotic actuator 10 in the robotic hand 100 has two chambers 208 and 209 for fluid injection. Each chamber is independently driven by a pressurized fluid. The chambers 208 and 209 are defined by two separated cavities 201 and 202 respectively. Similar to the robotic wrist 30 as described above, the chamber 209 is proximate to the patterned section 12 for limiting the bending angle of the flexible robotic actuator 10. The chamber 208 is illustrated to have a regular shape with a height, a width, and a length, despite other shapes are also possible, and it is used to control bending of the finger actuator 102 upon applying a fluid pressure P₁ (FIG. 7). The chamber 209 is with an undulated shape with a height, a width, and a length, despite other shapes are also possible, and it is used to control extension of the finger actuator 102 upon applying a fluid pressure P₂ (FIG. 8). In some embodiments, stiffness of the chamber 208 can be lower than that of the chamber 209, such that less pressure P₁ is already sufficient to bend the finger actuator 102 and larger pressure can be applied for actuator extension.

To control the movement of the finger actuator 102, pressurization of the chamber 208 with P₁ can facilitate bending toward chamber 209 to a flexed position 298 at 90 degree (FIG. 6B). On the other hand, pressurization of the chamber 290 with P₂ can facilitate extension toward the chamber 208 to an extended position 299 at 0 degree (FIG. 6C). The ring constraints 203 restrict the finger actuator 102 to further bend over by using the anchor structures 11 (i.e., less than 0 degree considering anticlockwise direction as positive in FIGS. 7 and 8). Therefore, two pressure sources may be applied to independently control the pressure inlets to the chambers 208 and 209, respectively, through two fluid channels or tubes 207 (such as rubber tube, PE tube, PVC tube, etc.). In case stiffness of the chamber 208 is lower than that of the chamber 209, pressure applied to the cavity 201 is inherently less than to the cavity 202. Between the chamber 208 and 209, there is formed with a gap 204 whose height may be no greater than 1 mm. An angle sensor 205 (such as flex sensor 4.5″, SparkFun Electronic) and a plate member (such as Polyethene, Nylon, etc.) with a thickness of no greater than 1 mm can be disposed within the gap 204 correspondingly. The angle sensor 205 returns a resistance change to an external electrical system as an indication of the actuator angle change. The finger actuator 102 may be molded or 3D printed with elastic materials, e.g., silicone, fabric, etc. In case of different stiffness between the chambers 208 and 209, the finger actuator 102 may be designed by co-molding or direct 3D printing, meaning that both chambers 208 and 209 can be adhered together without any extra assemble or gluing process in order to form the complete soft body.

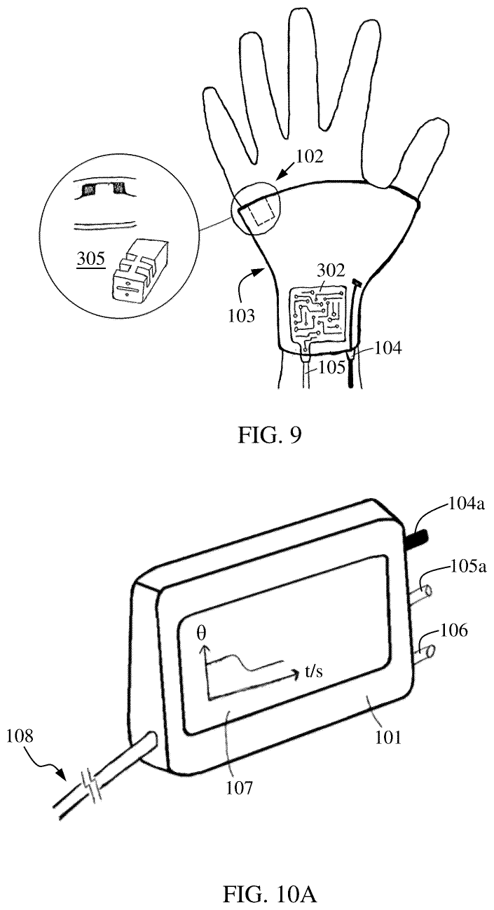

FIG. 9 illustrates an example internal structure of the hand base 103. The hand base 103 includes a control circuit 302 for routing the signals generated by the angle sensor 205, the inlet port 104, the data port 105, and a clamping structure 305 that enables the finger actuator 102 to be tightly secured to the hand base 103. The wearable hand base 103 includes materials that are biocompatible when contacting with human hand.

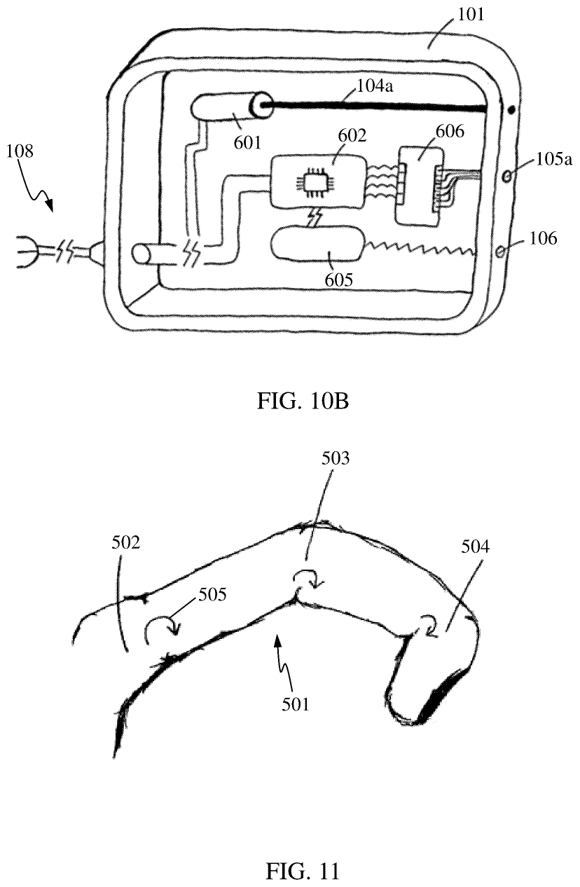

Referring to FIGS. 10A and 10B, a control system or module 101 is provided to communicate with a flexible robotic apparatus, such as a robotic wrist or hand. The control system 101 is in fluid communication with the at least one chamber of a flexible robotic actuator of the flexible robotic apparatus such that a pressurized fluid is injected into the chamber for deforming the flexible robotic actuator. The control system 101 is further in electrical communication with the flexible robotic apparatus such that operation of the flexible robotic actuator is electrically controlled.

By way of example, the control system 101 includes a fluid pump 601 (the pressure output may be no greater than 600 kPa), a computer device 602 (such as a mini PC tablet (e.g. Raspberry Pi)), an user interface or control panel 107, a power source 108 (such as a portable power source with an output voltage no greater than 24V), a simulation device or current simulator 605, and a data acquisition device 606. The user interface 107 displays a control program for users, e.g., clinicians, to control the usage of a flexible robotic apparatus, such as the robotic wrist 30 or the robotic hand 100. The fluid tube 104a and the data transmission cable 105a are adopted to connect the robotic hand 100 or robotic wrist 30 to the control system 101 for supplying fluid to the robotic hand 100 or robotic wrist 30 and receiving signal of bending angle through the flexible thin-film angle sensor 205. The electrode cable 106 is also adopted to allow the propagation of electric current from the simulation device 605 to human muscles, such as forearm muscles.

Referring to FIG. 11, it shows a compromised or disabled or flexed finger 501 (e.g., caused by stroke) with flexed Metacarpophalangeal (MCP) joint 502, Proximal Interphalangeal (PIP) joint 503, and Distal Interphalangeal (DIP) joint 504. The finger actuator 102 may be secured onto the flexed finger 501. It has been known that muscle spasticity is a major medical symptom (like joint pain, tendon fracture, etc.) occurred on patients with impaired hand function in which muscles stiffen or tighten, thereby preventing normal fluid movement of human tissues. The muscles remain contracted and resisted being stretched, thus affecting body joint movement. For patients with the flexed finger 501, spasticity has happened to finger flexor muscles, for which the muscles have contracted and created a resistance (or a torque) 505 to oppose the extension of finger joints. Spasticity has direct relationship with joint rotational speed, which higher speed will trigger larger resistance. The range of the torque 505 is typically located around 0.8 N-m to 1.7 N-m at different rotational speed. Orientation of the finger actuator 102, when worn on a human's finger, is different from the way when the robotic wrist 30 is worn on the human wrist 34. For the wearable robotic hand 100, the patterned section 12 proximate to the chamber 209 is proximally facing the back side of the finger 501. A bi-directional control, i.e., flexion and extension, is provided for the finger. Therefore, when the finger actuator 102 is secured on a spastic finger with actuation, the amount of extension torque that offered by the finger actuator 102 may be largely dependent on the elasticity of the soft body. Upon pressurization of the chamber 209 up to 600 kPa, a maximum extension torque of 1 N-m can be provided to counterbalance the flexion torque 505 due to muscle spasticity in finger joints for hand opening. Speed of the finger actuator 102 is controlled to be 6 degrees/second as well upon pressurization of the chamber 209 during extension. On the other hand, for patients with a disabled finger 501, spasticity happened to finger extensor muscles is uncommon, and therefore stiffness of the chamber 208 can be less than that of the chamber 209 for controlling the spastic finger 501. A maximum flexion torque of 0.5 N-m may be default for the finger actuator 102 upon pressurization of the chamber 208 up to 300 kPa. Speed of the finger actuator 102 may be controlled to be less than 6 degree/second as well upon pressurization of the chamber 208 during bending.

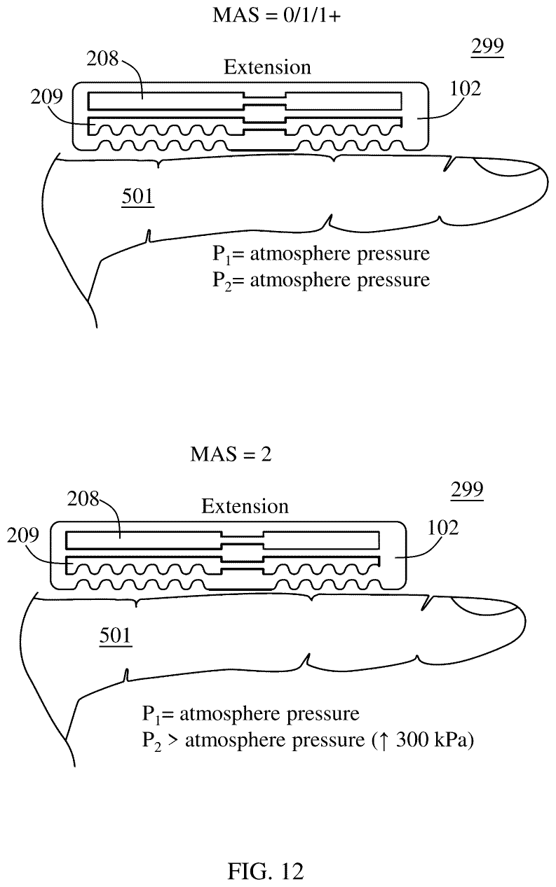

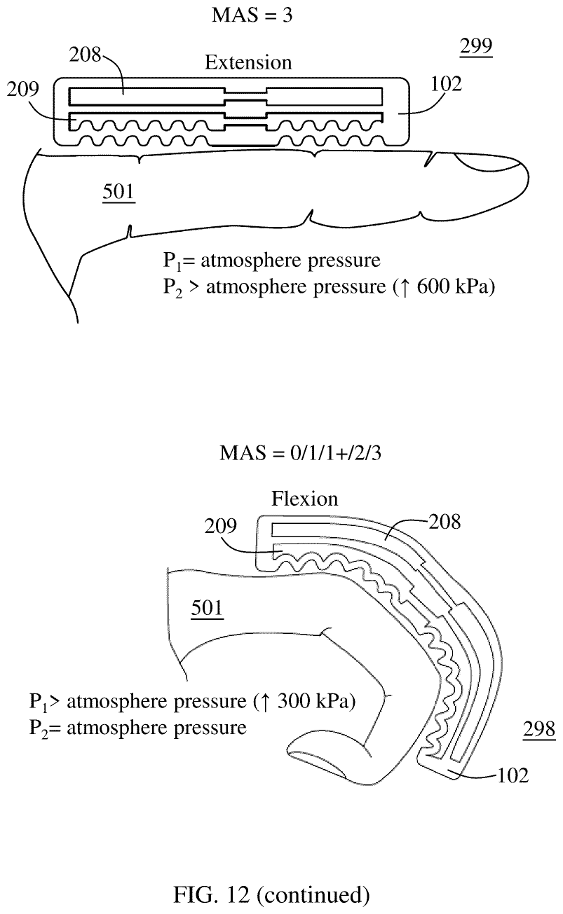

Referring to FIG. 12, depending on the severity of muscle spasticity assessed by clinicians for each patient, pressure output to the finger actuator 102 can be adjusted within the range of 0 kPa to 300 kPa for the chamber 208, and 0 kPa to 600 kPa for the chamber 209, such that the finger actuator can provide an improved or even optimal extension torque to straighten the spastic fingers 501. Instructions of pressure selection is referred to the score of Modified Ashworth Scale (MAS), which is a clinical assessment for grading the severity of spasticity from Grade 0=No increase in muscle tone; Grade 1=Slight increase in muscle tone at the end of the ROM; Grade 1+=Slight increase in muscle tone throughout less than half of the ROM; Grade 2=More marked increase in muscle tone throughout most of the ROM; Grade 3=Considerable increase in muscle tone throughout most of the ROM; Grade 4 =Complete rigid of the affected joint(s)).

In case of MAS=Grade 0, 1, 1+, since the muscle spasticity is not obvious, i.e., a neglectable flexion torque presented in the finger joints due to muscle spasticity, it is allowed that the bending and extension of a spastic finger is solely controlled by the chamber 208. When the finger actuator 102 is put on a disabled hand 501, finger flexion is controlled upon pressurizing the finger actuator to 300 kPa with a provided bending torque of 0.5 N-m. On the other hand, finger extension can be passively driven by the elasticity of material upon depressurizing the chamber 208 from 300 kPa to 0 kPa. The chamber 209 can remain in an unactuated state of 0 kPa all the time during bending and extension of the finger actuator 102.

In case of MAS=Grade 2, since the muscle spasticity become obvious, i.e., an obvious flexion torque presented in the finger joints due to muscle spasticity, both chambers 208 and 209 are required to be actuated. When the finger actuator 102 is put on a disabled hand 501, finger flexion is controlled upon pressurizing the finger actuator to 300 kPa with a provided bending torque of 0.5 N-m. On the other hand, finger extension is controlled upon pressurizing the chamber 209 to 300 kPa as well with a provided extension torque of 0.5 N-m.

In case of MAS=Grade 3, since the muscle spasticity become extremely severe, i.e., a very strong flexion torque presented in the finger joints due to muscle spasticity, both chambers 208 and 209 are required to be actuated as well. When the finger actuator 102 is put on a disabled hand 501, finger flexion is controlled upon pressurizing the finger actuator to 300 kPa with a provided bending torque of 0.5 N-m. On the other hand, finger extension is controlled upon fully pressurizing the chambers 209 to the maximum of 600 kPa as well with a provided extension torque of 1 N-m.

In case of MAS=Grade 4, since the finger joints become completely rigid in this case, no matter how to adjust the actuator 102, it can never be possible to control the flexion and extension of the spastic fingers. Therefore, the robotic hand 100 is not intended to be used on patients who are diagnosed with MAS=4 by clinicians.

The control scheme for the robotic wrist 30 is relatively simple, as only unidirectional control is required, i.e., wrist extension. Pressure applied to the chamber 15 is regulated depending on the grade of MAS as well.

In case of MAS=Grade 0, 1, 1+, a pressure up to 200 kPa is applied to control wrist extension, and a maximum torque of 1.7 N-m can be provided.

In case of MAS=Grade 2, a pressure up to 400 kPa is applied to control wrist extension, and a maximum torque of 3.4 N-m can be provided.

In case of MAS=Grade 3, a pressure up to 600 kPa is applied to control wrist extension, and a maximum torque of 5 N-m can be provided.

In case of MAS=Grade 4, since the wrist become completely rigid, no matter how to pressurize the chamber 15, it can never be possible to extend the wrist. Therefore, the robotic wrist 30 is also not intended to be used on patients who are diagnosed with MAS=4 by clinicians.

The above description regarding various MAS scenarios is for illustrative purpose only so as to elaborate certain applications of the flexible robotic actuators or finger actuators according to one or more embodiments. Based on the description, various variations are possible.

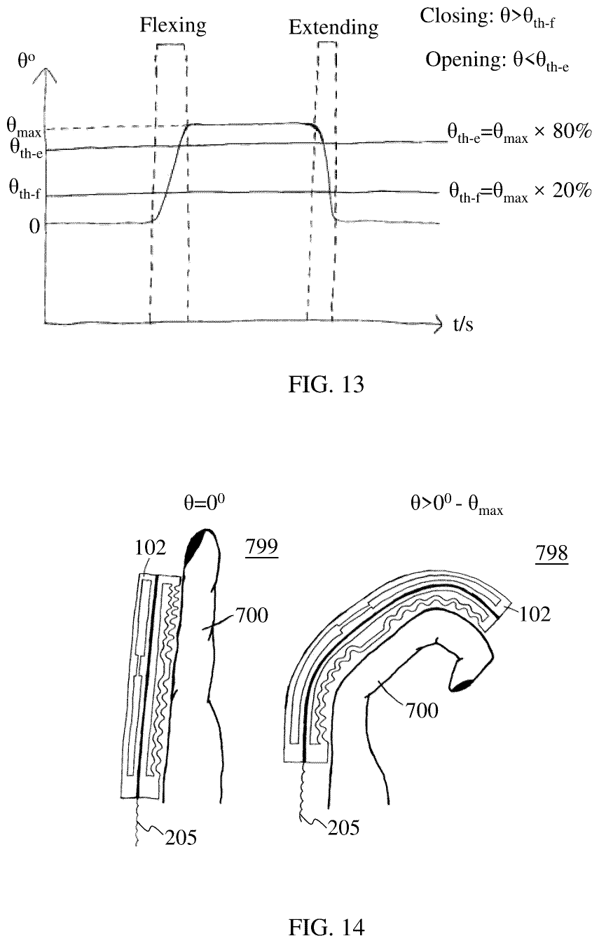

Referring to FIGS. 13 and 14, examples of capturing the subject's intention can be from the angle signals of the angle sensor 205 of a finger 700 or the wrist 34. In the present embodiments, the finger 700 is used for illustration. The angle sensor 205 is placed within the finger actuator 102 to measure the angle change. The subject voluntarily flexes the joint, e.g., the finger 700, from the extended position 799 to the flexed position 798. The angle sensor 205 measures the change of the bending angle during movement. When the measured bending angle is larger than the defined threshold for flexion θ_{th-f}, e.g., 20% of the magnitude θₘₐₓ in flexion during maximum voluntary contraction (MVC), the finger actuator 102 bends. When the bending angle is less than the defined threshold for extension θₜₕ₋ₑ, e.g., 80% of the magnitude θₘₐₓ in flexion during maximum voluntary contraction (MVC), the finger actuator 102 extends.

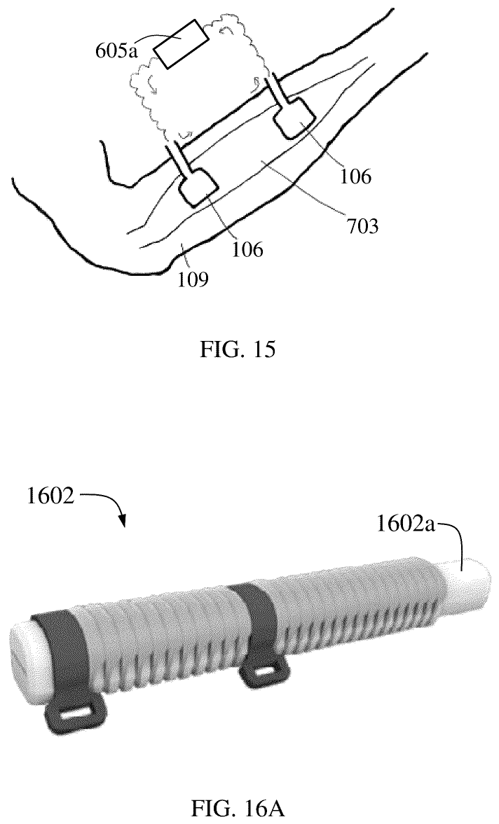

Further, for the subject with difficulties in finger or wrist extension, to assist with the movement, an electrical muscle stimulator 605a provides an electric current to stimulate the contraction of the subject's forearm muscles 703, e.g., Extensor Digitorium, through electrodes 106 attached on the forearm 109 as shown in FIG. 15.

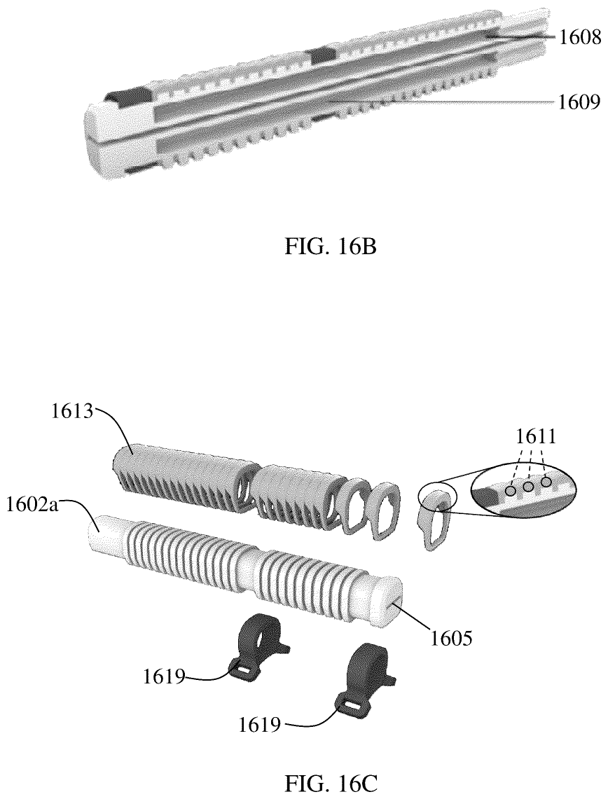

To further illustrate the spirits of the present invention, one or more soft actuators will be described below with reference to FIGS. 16A-24C. A soft actuator may be a flexible robotic actuator or a finger actuator as described above with reference to one or more embodiments, or one of their variations thereof.

Referring to FIGS. 16A, 16B, and 16C, the soft actuator 1602 includes a soft body 1602a and two chambers 1608 and 1609 defined by the soft body 1602a. For purpose of description, the chamber 1608 may be called a top chamber or top cavity, and the chamber 1609 may be called a bottom chamber or bottom cavity.

Further, ring constraints 1613 encloses or wraps the outer surface of the soft body 1602a to eliminate any irregular expansion of the chambers 1608 and 1609, and hence to facilitate the soft actuator's flexion and extension upon injection of the pressurized fluid. Anchor structures 1611 are provided on the ring constraints 1613 at one side of the soft body 1602a. The anchor structures 1611 help restrict over-extension beyond the top surface of the soft actuator 1602 when the bottom chamber 1609 is pressurized. On the opposite side of the soft body 1602a is provided with the patterned section (not shown). A flex sensor 1605 (such as a 4.5 inches angle sensor) is disposed within the soft body 1602a, and preferably between the two chambers 1608 and 1609 for measuring the bending angle of the soft body 1602a. Two holders 1619 (such as Velcro holders) facilitate attaching of the soft actuator 1602 onto a human hand.

The soft actuator 1602 is a bi-directional soft actuator as one chamber controls its flexion while the other controls its extension. Upon pressurizing the top chamber 1608, the soft actuator 1602 flexes towards the bottom chamber 1609, and vice versa upon pressurizing the bottom chamber 1609. Therefore, pneumatic sources can effectively control the flexion and extension, generating a much larger ROM. The soft actuator 1602 inherits the advantages of being lightweight, safe, and having a lower inherent impedance compared to its electric counterpart.

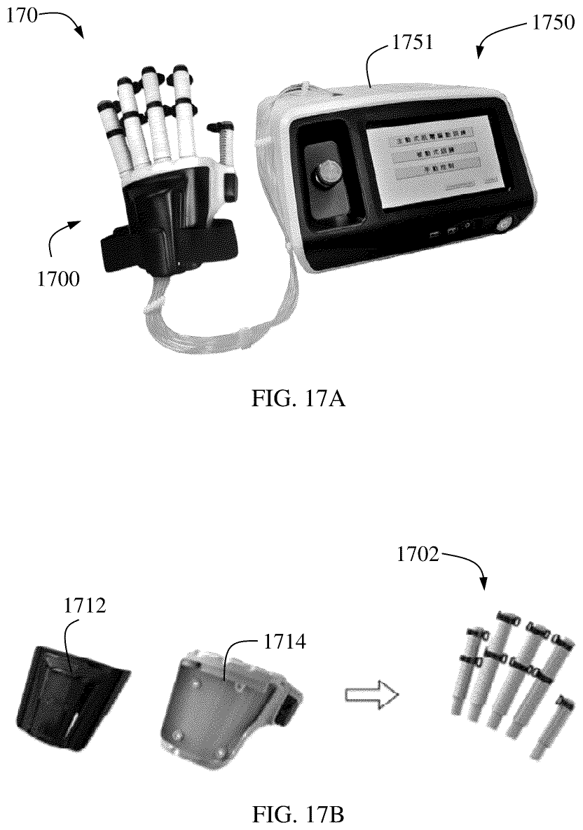

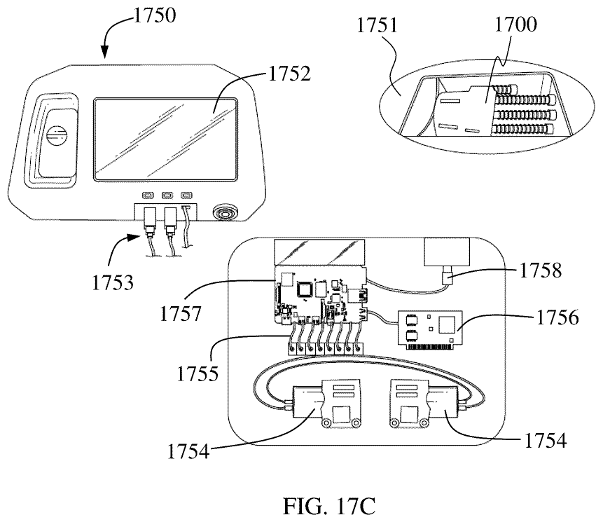

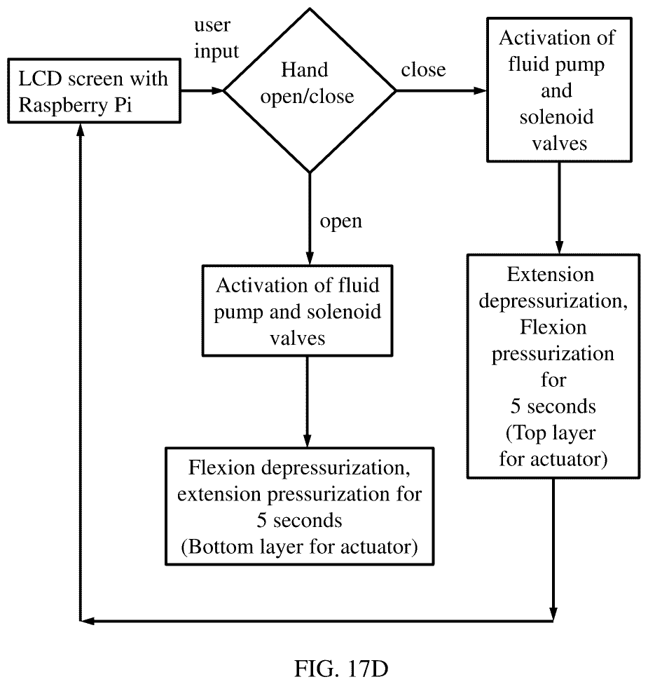

FIGS. 17A, 17B, 17C illustrate a flexible robotic system 170 according to certain embodiments of the present invention, where the flexible robotic system 170 includes a flexible robotic apparatus exemplified as a robotic hand 1700, and a control box 1750. FIG. 17D illustrates a control logic of the robotic hand 1700. The robotic hand 1700 can be the robotic hand 100 as described above according to one or more embodiments, or one of its variations.

The robotic hand 1700 includes five soft actuators 1702 and a soft base received in a housing consisting essentially of an outer shell 1712 and a base shell 1714 that can be assembled together. In some embodiments, the size of the soft actuator 1702 is 12 mm wide and 12 mm high. The length is 65 mm (for thumb), 85 mm (for small finger), or 105 mm (for the other three fingers). The size of the robotic hand 1700 is 17 cm (length)×10 cm (width)×3 cm (height). The weight of the five soft actuators 1702 and the robotic hand 1700 are 19 g and 176 g, respectively. These parameters are for illustrative purpose only. The size and weight of the soft actuators 1702 and the robotic hand 1700 can be designed differently according to practical needs.

The control box 1750 functions as a control system. It is provided with various pneumatic components, including a fluid pump 1754 (such as an air pump), solenoid valves 1755, air tubes and pressure sensors 1756, a LCD touchscreen 1757, an emergency button 1758, and other electronics, for controlling the pressure supplied to the soft actuators 1702. The emergency button 1758 is installed next to the control panel to immediately depressurize the soft actuators in case of emergency termination of usage. The control box 1750 has a control panel 1752 operatively to interact with users, and various electrical connections 1753. The LCD touchscreen 1757 presents the control panel 1752 to users, allowing them to control the system without connecting to computers. The control panel 1752 allows users to manually select the model of either hand closing or opening. On the top portion of the control box 1750 there is provided with a hand container 1751 configured to house the robotic hand 1700 for storage. This compact design makes the flexible robotic system 170 more portable. The control box 1750 may be 3D printed and sized to be 30 cm (length)×30 cm (width)×21 cm (height), and has a weight of 1.7 kg. These parameters are for illustrative purpose only. The size and weight of the control box 1750 can be designed differently according to practical needs.

Solenoid valves and fluid sources are either turned on or off by control signals for regulation of the pressure supplied to the soft actuators 1702. Pressure sensors are used to monitor the pressure supplied to the soft actuators 1702. Referring to FIG. 17D, upon selecting the hand closing option, the bottom layer of the soft actuators will be depressurized, and the top layer will be pressurized for 5 s for inflation of the chamber of each soft actuator, and vice versa in case of choosing the hand opening. Eventually, the Raspberry Pi records the joint angles measured and sends out the control signals to the solenoid valves for controlling the soft actuators, as well as to identify the output force with its mathematical model. This is for illustrative purpose only. The flexible robotic system 170 may be designed to operate in other ways according to practical needs.

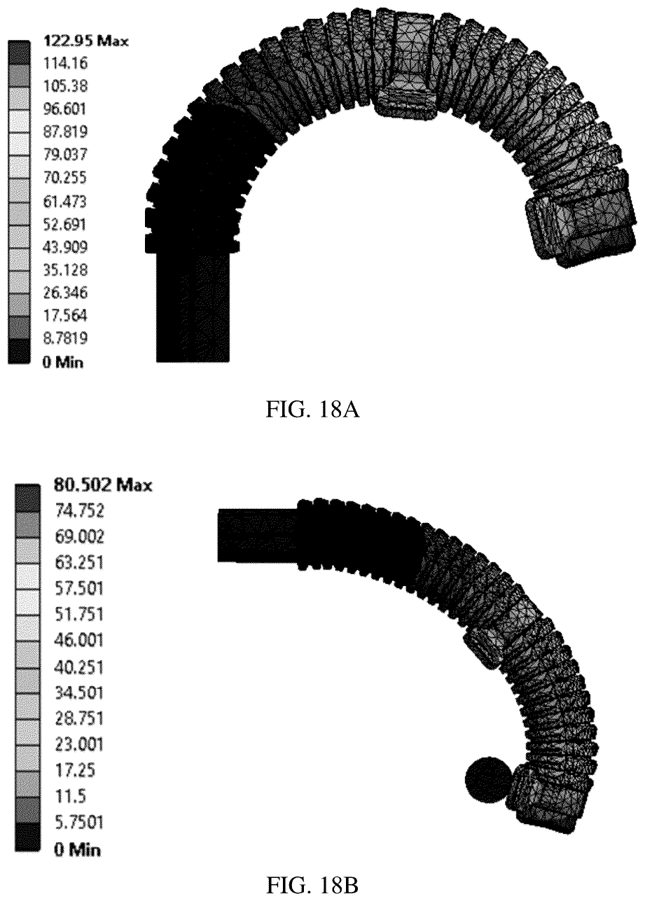

FIG. 18A and 18B show the FEM-simulated free space bending and contact force bending respectively for the soft actuator at pressure input of 300 kPa according to certain embodiments. The unit is millimetre (mm).

In this embodiment, specifically, ANSYS Workbench 15 is utilized to establish a 3D FEM model for the bi-direction soft actuator. The model is subjected to a Static Structural analysis to determine the bending angle and output force of the soft actuator under various input pressures. The setting of model is essentially the same as that was reported in the previous work: Heung, K. H. L., Tong, R. K. Y., Lau, A. T. H., and Li, Z. (2019a), Robotic glove with soft-elastic composite actuators for assisting activities of daily living, Soft Robot, 6 (2), 289-304, hereby incorporated herein by reference as to its entirety. The only one simplification made involves neglecting the pressure inlets and directly applying pressure to the internal chamber walls. To ensure accurate results, the 3D 10-Node tetrahedral structural solid elements (ANSYS element type SOLID187) are used for both the soft body (which is an elastomeric body in this embodiment) and the ring constraints, while the 3D 20-Node structural solid elements (ANSYS element type SOLID186) are used for the thin film flex sensor. An Ogden first order hyper-elastic model with the coefficients μ₁=75,449 Pa and α₁=5.836 is used to model Dragon Skin 30. For the ring constraints and flex sensor (which is Polyethylene for the simulation), the material properties are directly obtained from ANSYS Engineering Data Sources. The simulated results are shown in FIGS. 18A and 18B.

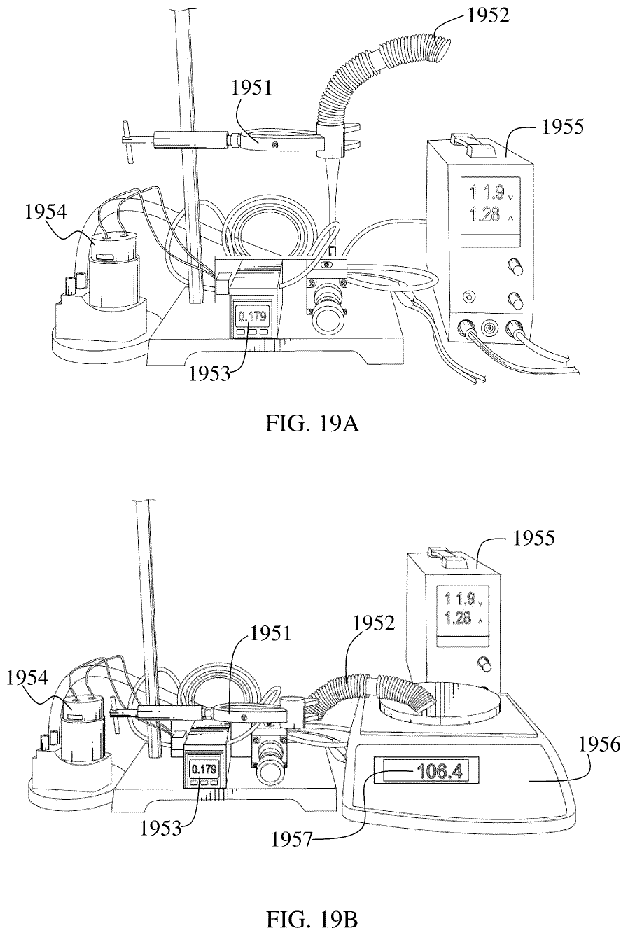

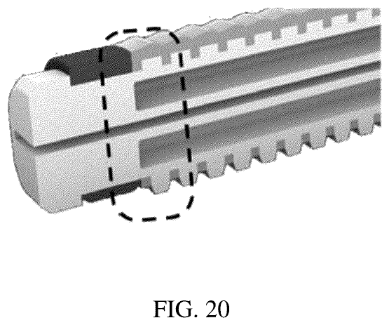

FIGS. 19A and 19B illustrate an example setup for actuator characterization regarding free space bending and contact force bending for a soft actuator respectively according to certain embodiments. The setup includes an air pump 1954 (which is an example of a fluid pump), a clamp 1951, a pressure meter 1953, a power source 1955, a soft actuator 1952, a load cell scale 1956 showing force readings 1957, and various other electronics and connections.

The bi-directional soft actuator 1952 is supplied with air pressure from the air pump 1954 (which, in this embodiment, is a BTC Diaphragm Pump, Parker Hannifin Corporation, Ohio, United States), which is controlled by the pressure meter 1953 [which, in this embodiment, is ZSE20C(F), SMC Pneumatic, Tokyo, Japan] and a pressure regulator (which, in this embodiment, is IR2020-02BG, SMC Pneumatic, Tokyo, Japan). The pressure regulator can be manually adjusted to control the air pressure supplied to the soft actuator 1952, and the pressure value is displayed on the screen of the pressure meter 1953. The power source 1955 is exemplified as a 12V voltage source that provide power to the system for operation.

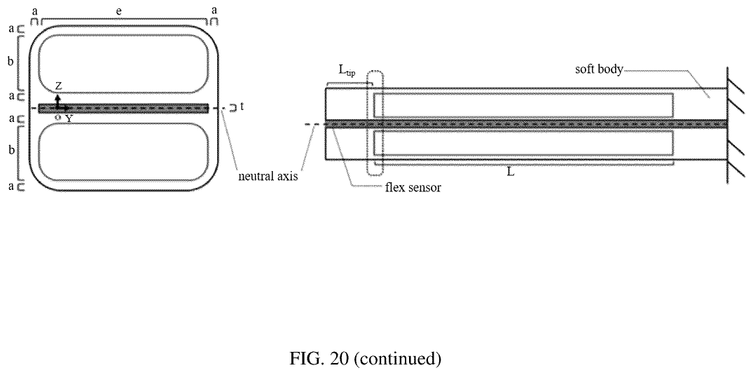

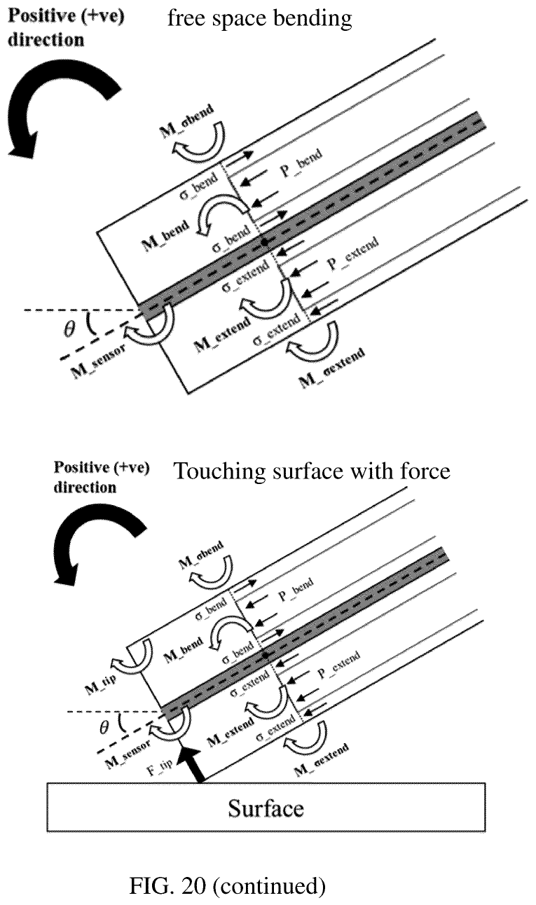

Referring to FIG. 20, a mathematical model is established for soft actuators, where a is wall thickness of the chamber of the soft actuator, b is the height of the chamber, e is the width of the chamber, L is the length of the chamber, t is the thickness of the flex sensor, Lₜᵢₚ is the length of the actuator tip. The model describes the correlation between the input pressures and the bending angle as well as the output force of the soft actuator. The model is static in nature, and considers the impact of the resistance created by the flex sensor and the soft body, as well as the bending moment that arises from the fluid injection into the chambers, in order to provide an accurate representation of the soft actuator.

When the pressure is applied, the soft actuator undergoes bending motion which is dependent on the pressure levels inside the two separate chambers. Assuming that the chambers are rectangular in shape and do not experience any cross-sectional deformation, the bending moment resulting from the pressure applied to each chamber can be determined by the following formulae.

M_{bend} represents moment for the actuator bending and can be expressed as

$${M}_{b e n d} = {\int }_{a + t / 2}^{b + a + t / 2} e \cdot {P}_{b e n d} \cdot z \cdot dz \qquad ( 1 )$$

M_{extend} represents moment for the actuator extension and can be expressed

$$as {M}_{extend} = {\int }_{- ( b + a + t / 2 )}^{- ( a + t / 2 )} e \cdot {P}_{extend} \cdot z \cdot dz \qquad ( 2 )$$

P_{bend} and P_{extend} represent the input pressure, and dz represents the differential height element in z-direction.

The bending of the soft actuator causes the soft body and the flex sensor to resist the bending deformation and creates a bending moment in the opposite direction to the bending itself. Further, in this embodiment, the bi-directional soft actuator is constructed using Dragon Skin 30 silicone rubber. This material can be described using an Ogden first order hyper-elastic model. The strain energy of the material is expressed

$$W ( {λ}_{1} , {λ}_{2} , {λ}_{3} ) = \frac{2 μ}{{α}_{1}^{2}} ( {λ}_{1}^{{α}_{1}} + {λ}_{2}^{{α}_{1}} + {λ}_{3}^{{α}_{1}} - 3 ) \qquad ( 3 )$$

The material coefficient α₁ is the strain hardening exponent, and μ is the small strain shear modulus. The internal stress σ_{bend} and σ_{extend} that oppose the bending deformation of soft actuator based on the Ogden material model can be expressed as

$${σ}_{b e n d} = {σ}_{extend} = {μ}_{1} ( {λ}^{{α}_{1}} - {λ}^{- {α}_{1}} ) \qquad ( 4 )$$

$$Here λ = \frac{θ z + L}{L} , and {μ}_{1} = \frac{2 μ}{{α}_{1}} \qquad ( 5 )$$

Here λ is the axial stretch along the soft actuator's length in x-direction, and

$${M}_{σ b e n d} = {\int }_{t / 2}^{a + t / 2} e \cdot {μ}_{1} ( {λ}^{{α}_{1}} - {λ}^{- {α}_{1}} ) \cdot z \cdot dz + 2 {\int }_{t / 2}^{b + 2 a + t / 2} a \cdot {μ}_{1} ( {λ}^{{α}_{1}} - {λ}^{- {α}_{1}} ) \cdot z \cdot dz + {\int }_{b + a + f / 2}^{b + 2 a + t / 2} e \cdot {μ}_{1} ( {λ}^{{α}_{1}} - {λ}^{- {α}_{1}} ) \cdot z \cdot dz \qquad ( 6 )$$

$${M}_{σ extend} = {\int }_{- ( a + t / 2 )}^{- t / 2} e \cdot {μ}_{1} ( {λ}^{{α}_{1}} - {λ}^{- {α}_{1}} ) \cdot z \cdot dz + 2 {\int }_{- ( b + 2 a + t / 2 )}^{- t / 2} a \cdot {μ}_{1} ( {λ}^{{α}_{1}} - {λ}^{- {α}_{1}} ) \cdot z \cdot dz + {\int }_{- ( b + 2 a + t / 2 )}^{- ( b + a + t / 2 )} e \cdot {μ}_{1} ( {λ}^{{α}_{1}} - {λ}^{- {α}_{1}} ) \cdot z \cdot dz \qquad ( 7 )$$

Mₛₑₙₛₒᵣ represents moment of the flex sensor and can be expressed as Mₛₑₙₛₒᵣ=

$$\frac{EI}{{L}_{s e n s o r}} \cdot θ \qquad ( 8 )$$

Here EI is the flexural rigidity of the flex sensor, where E is the elastic modulus and I is the second moment of area. Lₛₑₙₛₒᵣ is the length of the sensor. θ is the bending angle of the soft actuator. As an example, the flex sensor is a PE plastic film, and has a width of 6.35 mm and a length of 114.3 mm. Its elastic modulus and thickness are assumed to be 1 GPa and 1 mm.

When the proximal tip of the soft actuator is securely mounted, it will apply a force Fₜᵢₚ upon contact of its distal tip with an external object. This force is perpendicular to the bottom layer to maintain a constant bending moment arm of Lₜᵢₚ, which is the length of the actuator tip, relative to the fulcrum O. The exerted moment can be expressed as

$${M}_{tip} = {F}_{tip} \cdot {L}_{tip} \qquad ( 9 )$$

Here it is assumed that the force interaction happens at the end of the soft actuator, and the deformation along the soft actuator due to the force exertion is not accounted. Eventually, the response of bending angle to the input pressure can be found by the moment equilibrium achieved around the fulcrum O in bending. The integrals of the moment equilibrium can be solved numerically. For the free bending,

$${M}_{b e n d} + {M}_{extend} = {M}_{σ b e n d} - {M}_{σ extend} + {M}_{s e n s o r} \qquad ( 10 )$$

$$( {P}_{b e n d} - {P}_{extend} ) = \frac{2}{e b ( 2 a + t + b )} ( \frac{EI θ}{{L}_{s e n s o r}} + f ( θ ) ) \qquad ( 11 )$$

$$And f ( θ ) = {M}_{σ b e n d} - {M}_{σ extend} \qquad ( 12 )$$

For the touching object with contact force,

$${M}_{b e n d} + {M}_{extend} = {M}_{σ b e n d} - {M}_{σ extend} + {M}_{sensor} + {M}_{t i p} \qquad ( 13 )$$

$$( {P}_{b e n d} - {P}_{extend} ) = \frac{2}{e b ( 2 a + t + b )} \cdot ( \frac{EI θ}{{L}_{s e n s o r}} + f ( θ ) + {F}_{tip} {L}_{tip} ) \qquad ( 14 )$$

$$And f ( θ ) = {M}_{σ b e n d} - {M}_{σ extend} \qquad ( 15 )$$

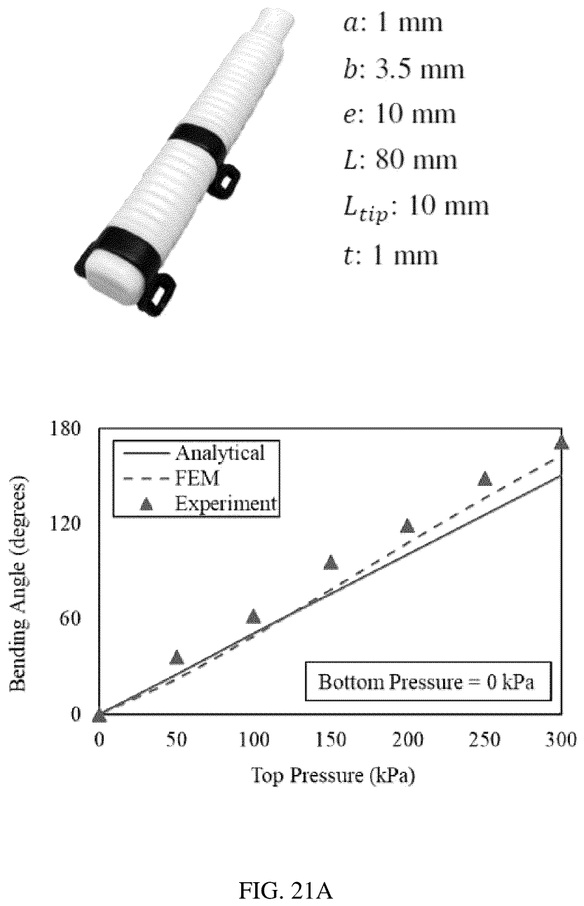

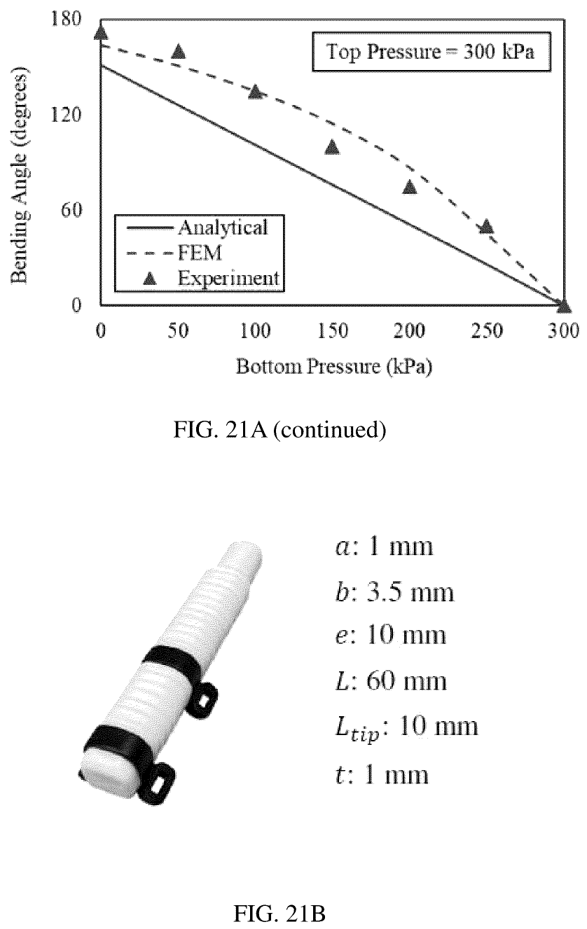

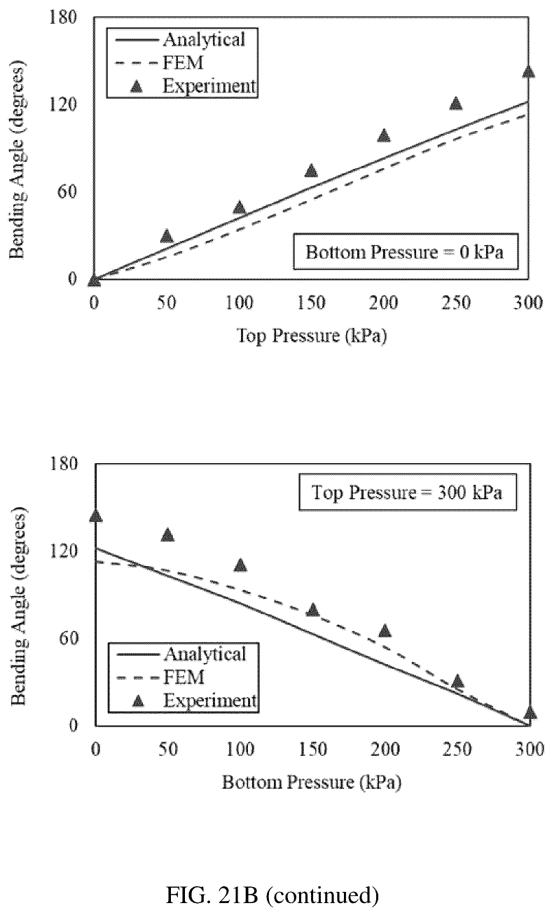

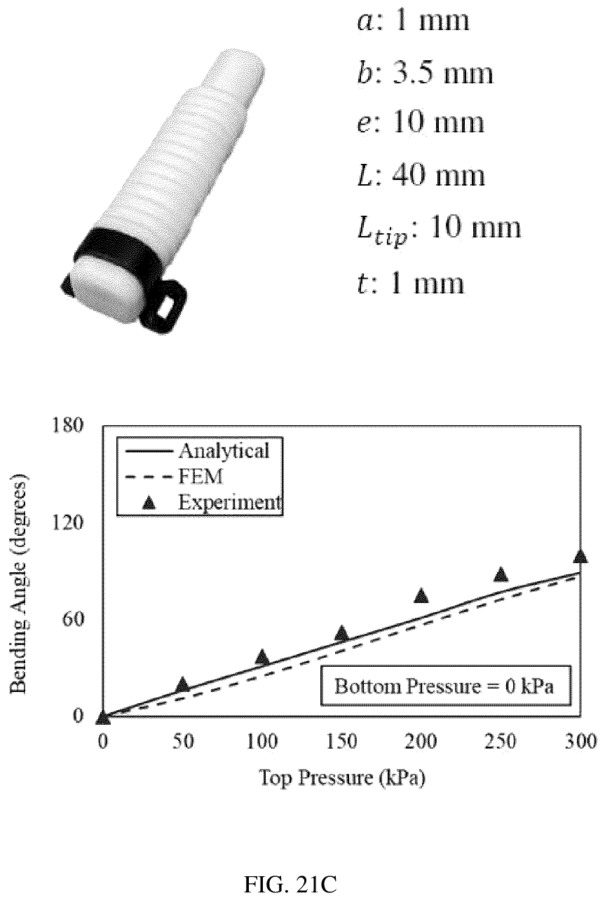

FIGS. 21A, 21B, and 21C show pressure-angle relationship for the soft actuators. The soft actuators are for index, middle, and ring fingers (FIG. 21A), a small finger (FIG. 21B), and a thumb (FIG. 21C) respectively. Each figure shows analytical results based on the mathematical mode as described above with reference to FIG. 20, FEM results, and experimental results using the experimental setup with reference to FIGS. 19A and 19B.

In these embodiments, the top bending chamber and bottom extension chamber are subjected to pressures ranging from 0 kPa to 300 kPa, with increments of 50 kPa. The resulting bending angles are then compared with those predicted by the analytical models and the FEM simulations. The flex sensor minimizes the effect of gravity on the bending angles, allowing for accurate measurements. The maximum input pressure for the soft actuator is limited to 300 kPa. As can be seen, the experimental results align well with both analytical models and FEM simulations, demonstrating that the bending angles increase with the length of the soft actuator at the same input pressure level. For instance, the actuator corresponding to the index, middle, and ring fingers achieve a bending angle of 172°, while the FEM simulation predict 164° and the analytical model predicted 151°. The maximum difference of 34° between the analytical model and experimental results is observed in the bending of the soft actuator corresponding to the three fingers at 300 kPa when the extension chamber is also pressurized to 100 kPa. The bi-directional soft actuator achieves actuator extension through different methods from that by prior art methods, such as in Shi, X. Q., Heung, H. L., Tang, Z. Q., Li, Z., and Tong, K. Y. (2021), Effects of a soft robotic hand for hand rehabilitation in chronic stroke survivors, J. Stroke Cerebrovasc. Dis. 30 (7), 105812. However, the bi-directional soft actuator as described herein according to one or more embodiments outperforms the prior art actuators by offering a larger ROM with less input pressure while maintaining the control over the actuator extension. The prior art actuators are unable to generate a large ROM without increasing input pressure, which reduces its lifespan and leads to rupture. That is, the soft actuators as described herein according to one or more embodiments achieve improved durability.

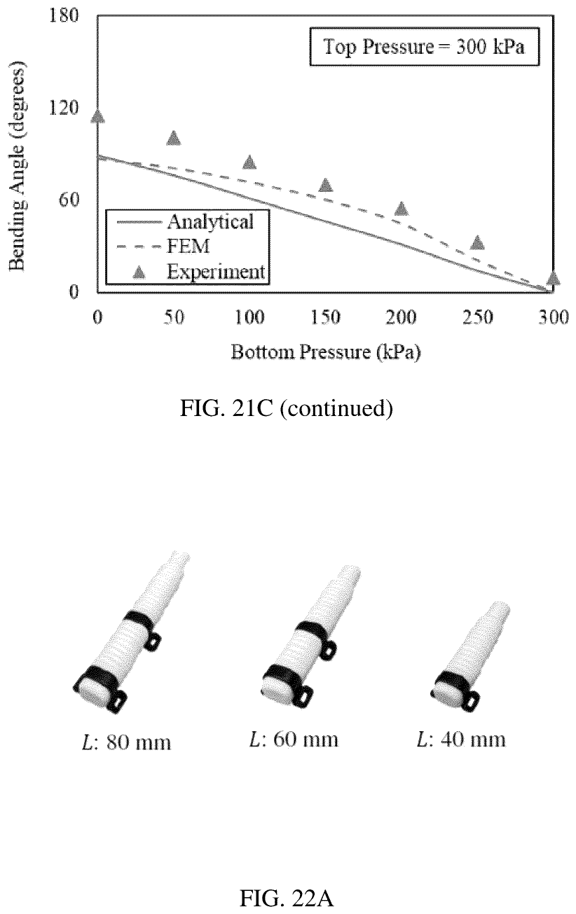

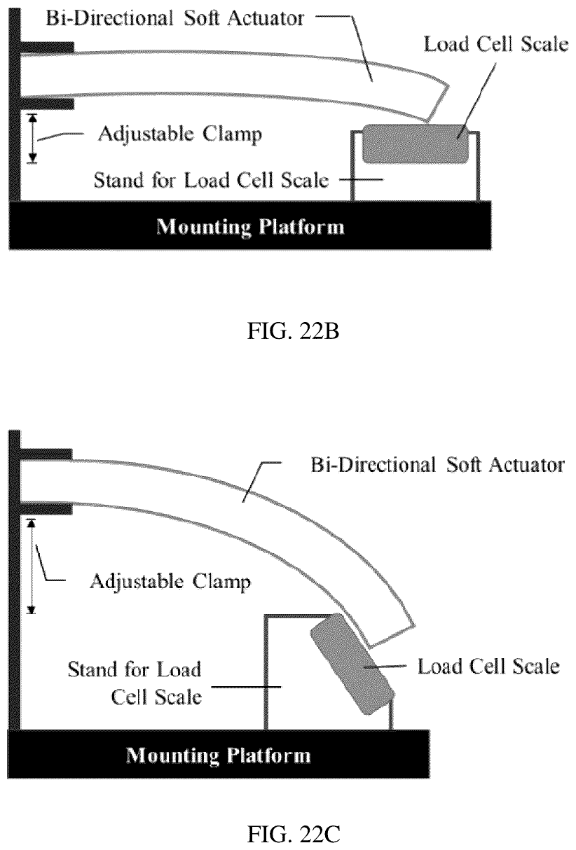

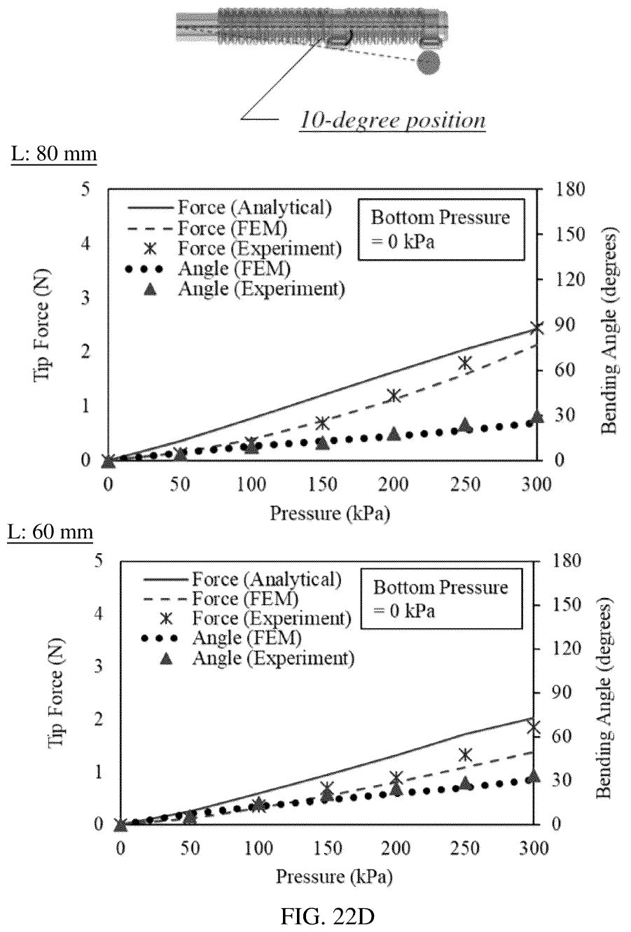

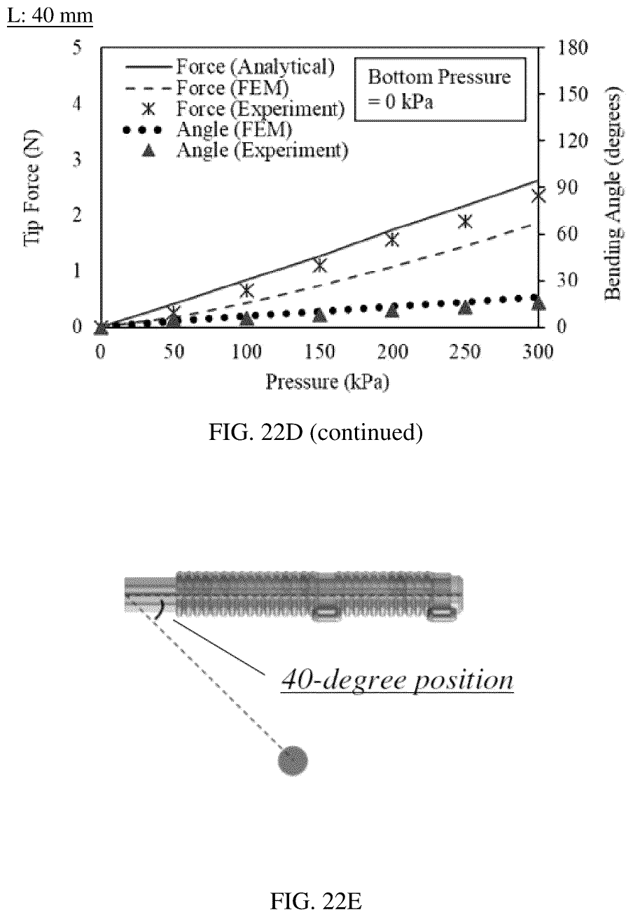

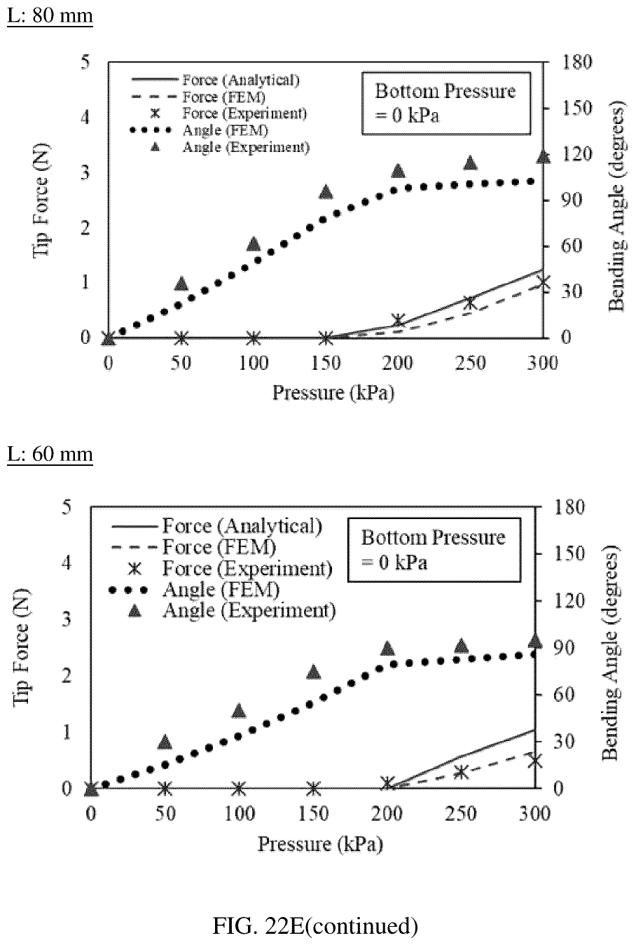

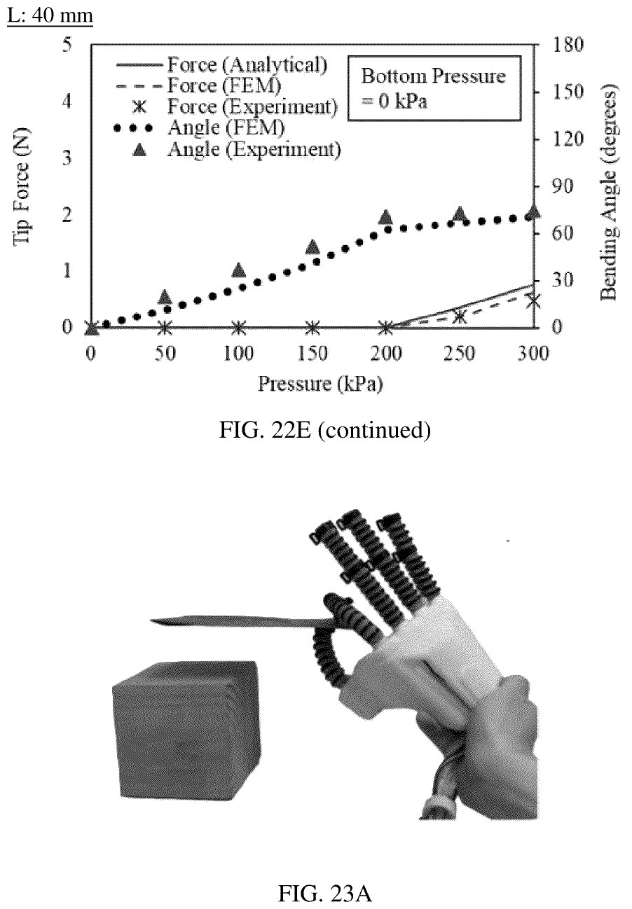

Referring to FIGS. 22A, 22B, 22C, 22D, and 22E, the pressure-force relationship for soft actuators with 10-degree and 40-degree angular positions are shown.

The top bending chamber is subjected to pressures ranging from 0 kPa to 300 kPa, with increments of 50 kPa. The resulting output force is then compared with those predicted by the analytical models and FEM simulations as well. The bending angles of the soft actuators are recorded during measurement, and the soft actuators will continue to bend (bulge) when subjected to increasing input pressure while in contact with objects. This bulging will affect the output force and should be taken into consideration. Furthermore, in order to calculate the output force of the actuator using analytical models and Equations (10) and (13), it is necessary to determine the bending angles of the soft actuators. To estimate the output force at the tip of the actuator, the bending angles obtained from FEM simulations are used in the models to calculate the analytical force when the actuators are under pressure and impeded by objects placed at 10-degree and 40-degree angular positions, respectively.

For the 10-degree angular position with respect to proximal end, the soft actuators experience bulging when they contact objects at bending angles of 30° (actuator representing the three fingers, FEM of 24.9°), 34° (actuator representing the small finger, FEM of) 30.9°, and 16° (actuator representing the thumb, FEM of 19.2°), respectively, when a pressure of 300 kPa is applied. The measured output force is 2.45 N (actuator representing the three fingers, FEM of 2.13 N, Analytical of 2.43 N), 1.85 N (actuator representing the three fingers, FEM of 1.38 N, Analytical of 2.03 N), and 2.36 N (actuator representing the three fingers, FEM of 1.87 N, Analytical of 2.63 N), respectively. The maximum difference of 0.52 N between the analytical and experimental results is observed on the actuator corresponding to the three fingers when pressurized to 150 kPa in the 10-degree position. No wall rupture or air leakage is observed during the tip force measurement.

For the 40-degree angular position with respect to the proximal end, The soft actuators experience insignificant bulging when they contact objects at bending angles of 119° (actuator representing the three fingers, FEM of 103.9°), 95° (actuator representing the small finger, FEM of 85.9°), and 75° (actuator representing the thumb, FEM of 70.5°), respectively, when a pressure of 300 kPa is applied. The measured output force is 1.02 N (actuator representing the three fingers, FEM of 0.97 N, Analytical of 1.24 N), 0.50 N (actuator representing the three fingers, FEM of 0.66 N, Analytical of 1.05 N), and 0.48 N (actuator representing the three fingers, FEM of 0.62 N, Analytical of 0.76 N), respectively. The maximum difference of 0.55 N between the analytical and experimental results is observed on the actuator corresponding to the small fingers when pressurized to 300 kPa.

The results of the experiment indicate that the stability of the soft actuator during grasping is influenced by the size of the grasped object. Specifically, the bulging effects are reduced as the bending angles increased, particularly when grasping smaller objects. The force estimation results are found to be close to linear when grasping larger objects but become more non-linear as the size of the objects and the length of the actuator increased. It is worth noting that the maximum possible flexion angle of the fingers is 180°, but a flexion angle of 137° is already sufficient for more than 90% of daily functional activities (Hume et al., 1990). Previous studies have reported that normal hand grasping generates fingertip forces ranging from around 0.25 N-3.59 N. Given these considerations, the output ROM and force from the soft actuators is considered to be sufficient for grasping and gripping most daily items such as bottles and cups.

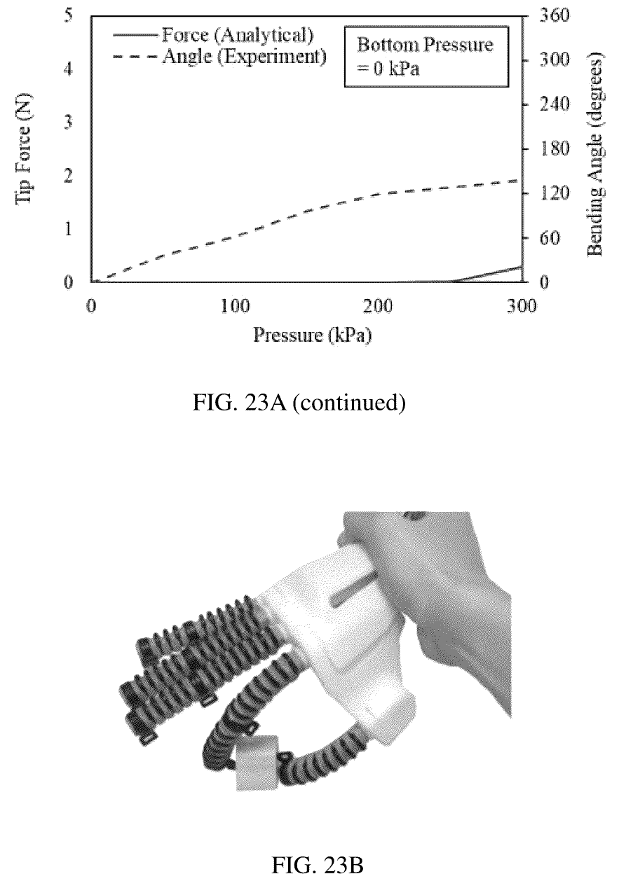

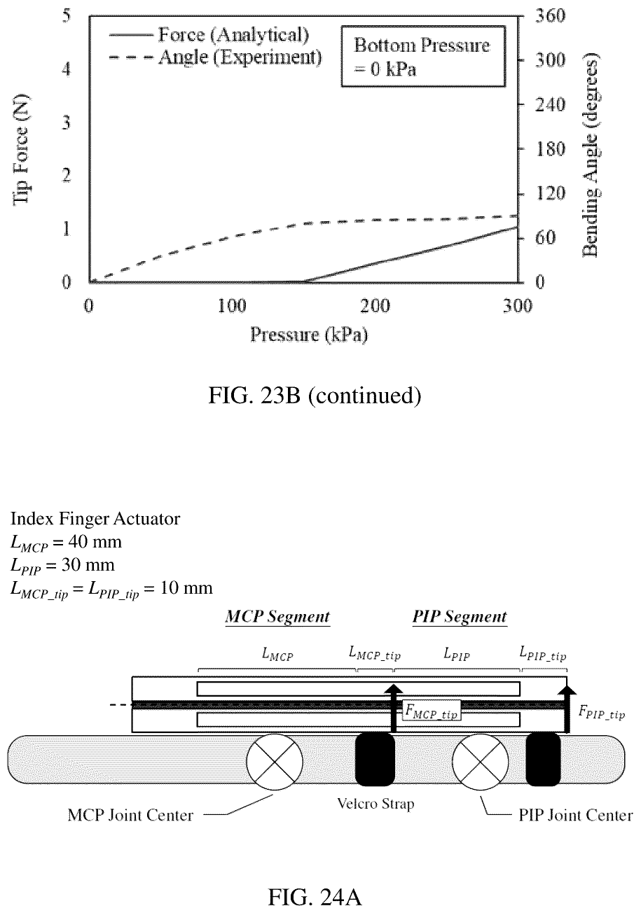

Referring to FIGS. 23A and 23B, it is shown the actuator tip force during the pinch of index finger for a card and (FIG. 23A) a wooden box (FIG. 23B) respectively.

The purpose of these tests is to evaluate the feasibility of using the soft robotic hand to assist with activities of daily living (ADL). It is tested the ability to grasp a card (length of 10 cm and width of 6 cm) and a wooden box (2.5×2.5×2.5 cm) without being worn on human hands. The bending angle of the soft actuator corresponding to the index finger is measured by flex sensors upon pressurization. The bending angle is then substituted into mathematical models based on Equations 13-15, along with the corresponding dimensions of the respective soft actuator, to estimate the grip force of the robotic hand when grasping objects. The robotic hand is manually controlled as an indicator to the subject, and a constant pressure of 300 kPa is applied during each actuation step. While the soft actuator corresponding to the index finger is selected for the evaluation of tip force upon pinching of objects, it is estimated that the pinch force at index finger achieved 0.285 N and 1.05 N for the card and the wooden box respectively at 300 kPa. As a result, the force estimated by the mathematical models of the soft actuators (FIGS. 23A and 23B) is found to be in agreement with previous research conducted for measuring fingertip force during object grasping, within around 1 N-2 N (see Yap, H. K., Lim, J. H., Nasrallah, F., Goh, J. C. H., and Yeow, R. C. H. (2015), “A soft exoskeleton for hand assistive and rehabilitation application using pneumatic actuators with variable stiffness,” in Proceeding of the IEEE International Conference on Robotics and Automation (ICRA), May 2015 (Seattle, WA, USA: IEEE), 4967-4972). This validation test has demonstrated that the soft robotic hand is capable of grasping objects with sufficient force for ADL-related tasks.

To further evaluate the effectiveness of the soft robotic hand, two healthy subjects (age: 28, male; age: 26, female) with intact hand function from the inventors' research team are recruited with informed consent. During the evaluation, the subjects are required to remain relaxed to avoid influencing the bending performance of actuators and ensure optimal estimation of output force.

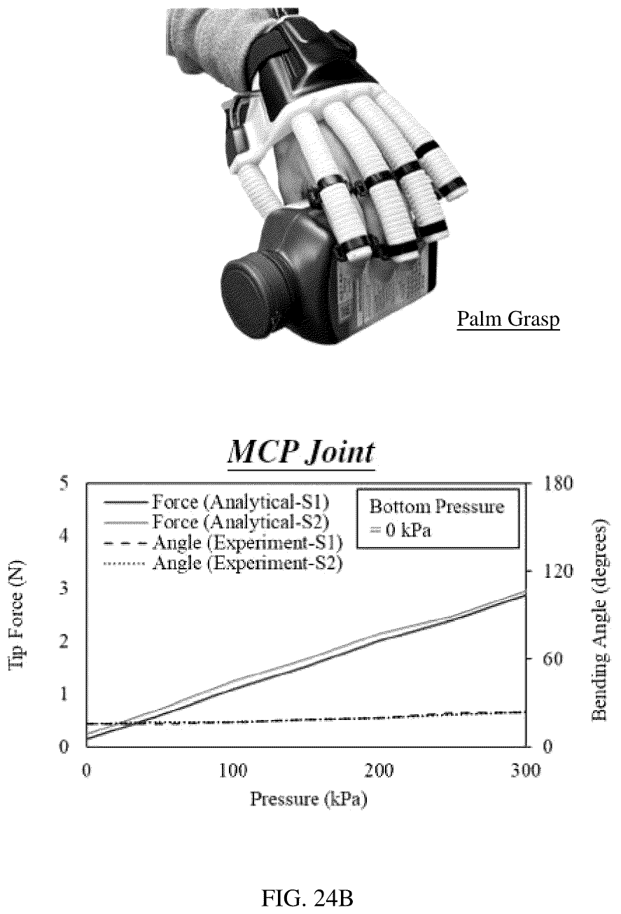

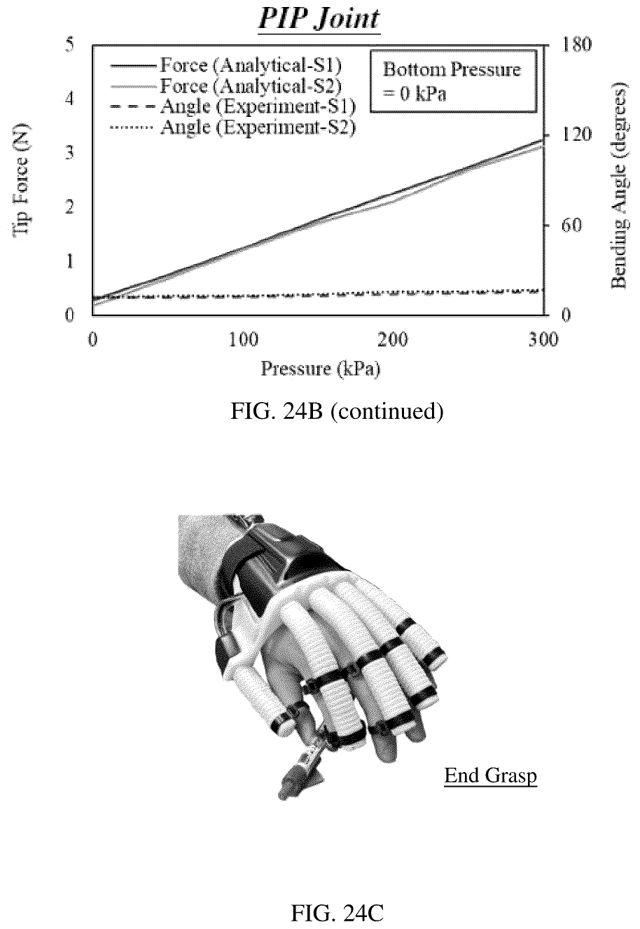

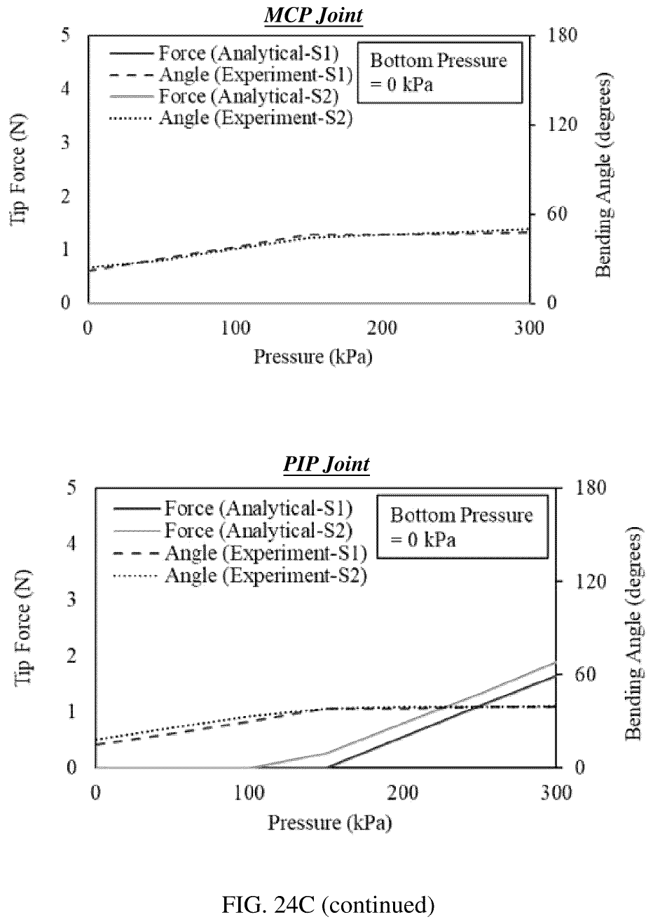

Referring to FIGS. 24A, 24B, and 24C, to apply the analytical models based on Equations 13-15 that estimate the fingertip contact force during the grasp of objects, it is considered the torque of finger joints that influences the bending of the soft actuators upon pressurization. Assuming the gap existed between the soft actuator and the finger to be ignorable, the kinematics of fingers can be represented by

$${M}_{joint} = {k}_{joint} ( {θ}_{rest} - θ ) ; θ < {θ}_{rest} \qquad ( 16 )$$

Here kⱼₒᵢₙₜ is the finger joint stiffness, θ is the joint angle, and θᵣₑₛₜ is the initial resting angle when there is no exerted voluntary movement, as human fingers tend to curl inwards and remain in a flexed position (θᵣₑₛₜ) due to the muscle tone naturally presented in finger flexors (e.g., flexor digitorum profundus) being larger than that of finger extensors (e.g., extensor digitorum).

The bi-directional soft actuator may be considered as equivalent to being composed of two segments, i.e., MCP and PIP segments, respectively (FIG. 24A). While each segment of the soft actuator is covering the MCP and PIP joints during the grasp of objects, the bottom cavity inside the soft actuator remains unpressurized.

Regarding MCP segment when touching objects,

$${M}_{MCP\_bend} = {M}_{σ MCP\_bend} - {M}_{σ MCP \_ extend} + {M}_{MCP\_sensor} + {M}_{MCP\_tip} + {M}_{MCP\_joint} \qquad ( 17 )$$

$${P}_{{MCP}_{- bend}} = \frac{2}{e b ( 2 a + t + b )} \cdot ( \frac{EI θ}{{L}_{{MCP}_{\_ sensor}}} + f ( θ ) + {F}_{{MCP}_{\_ tip}} {L}_{{MCP}_{\_ tip}} + {k}_{{MCP}_{\_ joint}} ( {θ}_{MCP} - {θ}_{MCP\_rest} ) \qquad ( 18 )$$

$$And f ( θ ) = {M}_{σ MCP\_bend} - {M}_{σ MCP\_extend} \qquad ( 19 )$$

For PIP segment when touching objects,

$${M}_{PIP\_bend} = {M}_{σ PIP\_bend} - {M}_{σ PIP\_extend} + {M}_{PIP\_sensor} + {M}_{PIP\_tip} - {M}_{PIP\_joint} \qquad ( 20 )$$

$${P}_{{PIP}_{- bend}} = \frac{2}{e b ( 2 a + t + b )} \cdot ( \frac{EI θ}{{L}_{{PIP}_{\_ sensor}}} + f ( θ ) + {F}_{{PIP}_{\_ tip}} {L}_{{PIP}_{\_ tip}} + {k}_{PIP\_joint} ( {θ}_{PIP} - {θ}_{PIP\_rest} ) \qquad ( 21 )$$

$$And f ( θ ) = {M}_{σ PIP\_bend} - {M}_{σ PIP\_extend} \qquad ( 22 )$$

In the experiment, the soft actuator corresponding to the index finger is selected for the preliminary evaluation of grip force estimation based on the modeling Equations 17-22. Bending angles of the MCP and PIP segments are measured by flex sensors upon pressurization. For the male subject (“S1” thereinafter), θ_{MCP_rest}=46°, and θ_{PIP_rest}=40°. For the female subject (“S2” thereinafter), θ_{MCP_rest}=58°, and θ_{PIP_rest}=49°. For both subjects, K_{MCP_joint}=0.01876, and K_{PIP_joint}=0.01533. The robotic hand is manually controlled as an indicator to the subject, and a constant pressure of 300 kPa is applied during each actuation step. Unilateral tasks involving the palm grasp of a bottle (9×7 ×15 cm) and the end grasp of a pen (radius of 1 cm and length of 14 cm) are further assigned to the subjects. The two subjects have successfully performed both tasks while wearing the robotic hand (FIGS. 24 B and 24C). No active voluntary movement of finger flexion and extension are allowed throughout the whole process. From the results of grasping the bottle, an estimated grip force of 0.15 N and 0.29 N (S1) and 0.25 N and 0.18 N (S2) are naturally presented at both MCP and PIP joint positions prior to robotic hand actuation. They naturally grasp the bottle even without any voluntary movement due to the natural contraction of finger flexors while putting their hands to the bottle. When the maximum pressure of 300 kPa is applied, an estimated grip force of 2.88 N and 2.96 N (S1) and 3.25 N and 3.13 N (S2) is obtained. On the other hand, since the MCP joint is not involved at all during the end grasp, only the grip force at PIP joint position is considered when grasping the pen. When the input pressure reaches 100 kPa, the soft actuator flexes the index finger of S2 touching the pen, and the same for S1 at 150 kPa. A maximum grip force of 1.64 N (S1) and 1.89 N (S2) are estimated at 300 kPa of input pressure. It is noted that the assisted grip force is not constant under the same input pressure and depends on the size of the objects. To grasp an object, smaller size will require a larger actuator bending angle to flex the fingers to the position of the objects, which directly reduces the output force provided for the grasp of smaller objects.

As used herein, wherever something is modified by the term “soft” or “flexible”, that means said something is composed essentially of compliant materials, rather than rigid materials.

It will further be appreciated that any of the features in the above embodiments of the invention may be combined together and are not necessarily applied in isolation from each other. Similar combinations of two or more features from the above described embodiments or preferred forms of the invention can be readily made by one skilled in the art.

Unless otherwise defined, the technical and scientific terms used herein have the plain meanings as commonly understood by those skill in the art to which the example embodiments pertain. Embodiments are illustrated in non-limiting examples. Based on the above disclosed embodiments, various modifications that can be conceived of by those skilled in the art would fall within spirits of the example embodiments.

What is claimed is:

1. A flexible robotic actuator for assisting a body part of a subject, the flexible robotic actuator comprising:
a soft body having a first side and a second side opposite the first side, the soft body including a patterned section on the first side; and
at least one chamber defined by the soft body and operatively driven by a pressurized fluid such that the soft body bends towards the patterned section in a first direction and the bending angle of the soft body is limited by the patterned section.

2. The flexible robotic actuator of claim 1, wherein the patterned section has a zigzag pattern.

3. The flexible robotic actuator of claim 1, further comprising a plurality of ring constraints provided for the soft body and surrounding the at least one chamber for restricting axial expansion of the at least one chamber when the at least one chamber is driven by the pressurized fluid.

4. The flexible robotic actuator of claim 3, wherein the stiffness of the plurality of ring constraints is higher than the stiffness of the soft body, and the plurality of ring constraints is made of one or more materials selected from a group consisting of titanium alloy, nylon, plastic, and carbon fibre composites.

5. The flexible robotic actuator of claim 3, wherein the plurality of ring constraints are provided with a plurality of anchor structures on the second side of the soft body for limiting bending of the soft body towards a second direction opposite the first direction.

6. The flexible robotic actuator of claim 1, wherein the bending angle of the soft body is in a range from 0 degree to 90 degree.

7. The flexible robotic actuator of claim 1, further comprising a plate member embedded within the soft body and disposed between the patterned section and the at least one chamber for assisting the bending of the soft body towards the patterned section.

8. The flexible robotic actuator of claim 7, wherein the plate member has a thickness of less than 1 mm.

9. The flexible robotic actuator of claim 7, wherein the plate member is made of one or more materials selected from a group consisting of plastic, metal, and paper.

10. The flexible robotic actuator of claim 3, further comprising an elastic sleeve for enclosing at least a part of the soft body along the longitudinal direction of the soft body for improving securing of the plurality of ring constraints onto the soft body.

11. The flexible robotic actuator of claim 10, wherein the durometer of the elastic sleeve is smaller than the durometer of the soft body such that the elastic sleeve accommodates deformation of the soft body.

12. The flexible robotic actuator of claim 1, wherein the at least one chamber includes a first chamber and a second chamber, each chamber being independently driven by the pressurized fluid.

13. The flexible robotic actuator of claim 12, wherein the first chamber is operatively driven to generate a bending force exerted upon the body part of the subject, and the second chamber is operatively driven to generate an extension force exerted upon the body part.

14. The flexible robotic actuator of claim 13, wherein the bending force and the extension force are configured to control flexion and extension of a proximal interphalangeal (PIP) joint and a metacarpophalangeal (MCP) joint of the subject, respectively.

15. The flexible robotic actuator of claim 1, further comprising an angle sensor configured to measure the bending angle of the soft body.

16. The flexible robotic actuator of claim 15, wherein the angle sensor is a flexible thin-film flex sensor extending along the longitudinal axis of the soft body.

17. The flexible robotic actuator of claim 1, wherein the soft body is an elastomer body.

18. A flexible robotic apparatus for assisting a body part of a subject, comprising:
a soft base wearable onto the body part of the subject; and
at least one flexible robotic actuator according to claim 1,
wherein the at least one flexible robotic actuator is configured to be secured to the soft base, and the soft base includes at least one fluid inlet in fluid communication with the at least one chamber for receiving the pressurized fluid from an external fluid source, and at least one data port electrically communicating with an external electrical system such that at least one parameter associated with the at least one flexible robotic actuator is monitored.

19. The flexible robotic apparatus of claim 18, wherein the at least one parameter includes the bending angle of the at least one flexible robotic actuator.

20. The flexible robotic apparatus of claim 18, wherein the flexible robotic apparatus is a robotic wrist, and the soft base includes a wrist band for wrapping around a wrist of the subject.

21. The flexible robotic apparatus of claim 18, wherein the flexible robotic apparatus is a robotic hand.

22. The flexible robotic apparatus of claim 21, wherein the at least one flexible robotic actuator forms five finger actuators with each finger actuator configured to be used for a respective finger of the subject.

23. The flexible robotic apparatus of claim 22, wherein each of the five finger actuators includes two flexible robotic actuators connected in series.

24. The flexible robotic apparatus of claim 22, wherein each of the five finger actuators includes a first chamber and a second chamber, each chamber being independently driven by the pressurized fluid,
wherein the first chamber is operatively driven to generate a bending force exerted upon the corresponding finger, and the second chamber is operatively driven to generate an extension force exerted upon the corresponding finger.

25. The flexible robotic apparatus of claim 24, wherein each of the five finger actuators is provided with a flex sensor disposed between the first chamber and the second chamber for measuring the bending angle of the corresponding finger actuator.

26. A flexible robotic system for assisting a body part of a subject, comprising:
a flexible robotic apparatus including a soft base wearable onto the body part of the subject and at least one flexible robotic actuator configured to be secured to the soft base, each of the at least one flexible robotic actuator including a soft body and at least one chamber defined by the soft body, the soft body including a patterned section on one side and configured to operatively bend towards the patterned section when the at least one chamber is driven by a pressurized fluid; and
a control system in fluid communication with the at least one chamber of each of the at least one flexible robotic actuator for controlling injection of the pressurized fluid into the at least one chamber, and in electrical communication with the flexible robotic apparatus such that operation of the at least flexible robotic actuator is electrically controlled.

27. The flexible robotic system of claim 26, wherein each of the at least one flexible robotic actuator includes an angle sensor configured to measure the bending angle of the soft body, and the control system is configured to receive an angle signal from the angle sensor indicating the bending angle.

28. The flexible robotic system of claim 26, wherein the control system includes a fluid pump and a solenoid valve configured to regulate the pressurized fluid into the at least one chamber to deform the soft body.

29. The flexible robotic system of claim 26, wherein the control system includes an electrical muscle stimulator configured to generate electric current to muscles of the subject.

30. The flexible robotic system of claim 29, wherein the electric current stimulates the contraction of forearm muscles of the subject.

31. A method for assisting a body part of a subject, the method comprising:
providing a flexible robotic apparatus, the flexible robotic apparatus including a soft base wearable onto the body part of the subject and a flexible robotic actuator, the flexible robotic actuator being secured to the soft base, the flexible robotic actuator including a soft body and at least one chamber defined by the soft body, the soft body including a patterned section on one side and being provided with a plurality of ring constraints surrounding the at least one chamber, the plurality of ring constraints being provided with a plurality of anchor structures on the other side of the soft body opposite to the one side; and
injecting a pressurized fluid into the at least one chamber such that the soft body bends towards the patterned section and the bending angle of the soft body is limited by the patterned section.

32. The method of claim 31, further comprising controlling the pressure of the pressurized fluid within the at least one chamber by adjusting the flow rate of the pressurized fluid into the at least one chamber.

33. The method of claim 31, further comprising generating electric current for stimulating muscles of the subject.

34. The method of claim 31, wherein the at least one chamber includes a first chamber and a second chamber, and the method further comprises:
driving the first chamber by the pressurized fluid to generate a bending force exerted upon a finger of the subject; and
driving the second chamber by the pressurized fluid to generate an extension force exerted upon the finger of the subject.

35. The method of claim 31, wherein providing the flexible robotic apparatus comprising providing a robotic wrist or a robotic hand as the flexible robotic apparatus.
